(19) United States
(12) Patent Application Publication
(10) Pub. No.: US 2024/0374546 A1
(43) Pub. Date: Nov. 14, 2024

(54) USES AND METHODS FOR PROMOTING INCREASED MITOCHONDRIAL MASS AND FUNCTION

(71) Applicants: Brightseed, Inc., South San Francisco, CA (US); Sanford Burnham Prebys Medical Discovery Institute, La Jolla, CA (US)

(72) Inventors: Lee Heil Chae, South San Francisco, CA (US); Fred Levine, La Jolla, CA (US)

(21) Appl. No.: 18/775,510

(22) Filed: Jul. 17, 2024

Related U.S. Application Data
(63) Continuation of application No. PCT/US2023/060772, filed on Jan. 17, 2023.
(60) Provisional application No. 63/300,364, filed on Jan. 18, 2022.

(51) Int. Cl. A61K 31/165; A61K 9/00; A61P 1/16; A61P 3/00

(52) U.S. Cl. CPC A61K 31/165; A61K 9/0053; A61P 1/16; A61P 3/00

(57) ABSTRACT

Disclosed herein are methods for promoting increased mitochondrial mass and function by providing a consumable composition. Some embodiments provided include, for example, administering a compound of Formula (I) or compound of Formula (II). Some embodiments provide the composition is formulated as a dietary supplement, food ingredient or additive, a medical food, nutraceutical or pharmaceutical composition.

### CROSS-REFERENCE TO RELATED APPLICATIONS

This application is a continuation of U.S. National Phase of Internation No. PCT/US2023/060772, filed Jan. 17, 2023, and claims the benefit of U.S. Provisional Application No. 63/300,364, filed Jan. 18, 2022, which is incorporated by reference in its entirety.

### BACKGROUND

HNF4α is a nuclear receptor transcription factor that controls the expression of downstream genes that are important in multiple aspects of cellular metabolism. The classical view of HNF4α, has been that its ligand binding pocket (LBP) is constitutively occupied by a fatty acid that plays a structural rather than regulatory role. However, it has been shown recently that the fatty acids in the HNF4α LBP are exchangeable in the context of full length HNF4α, particularly inside the cell.

### SUMMARY OF THE DISCLOSURE

Disclosed herein are methods for promoting mitochondrial mass and function in a subject in need. In some embodiments, the method includes administering to the subject in need thereof an oral composition comprising at least one carrier and an effective amount of N-trans-caffeoyltyramine, wherein the subject is on a high fat diet.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1A illustrates liver sections from mice fed normal chow (NC), high fat diet (HFD) or HFD+NCT were immunostained for HNF4α (green nuclear staining) and DAPI (blue nuclear staining). FIG. 1B illustrates quantification of HNF4α fluorescence intensity from images stained as in FIG. 1A. Non-specific cytoplasmic staining was subtracted from the HNF4α nuclear staining and fold change was calculated vs. NC (NC, N=3, HFD and HFD+NCT, N=12). FIG. 1C illustrates body weight was measured each week for 10 weeks. Body weight gain was calculated by subtracting the baseline body weight at the start of the study (NC N=5, HFD and HFD+NCT, N=15). FIG. 1D illustrates HFD and HFD+NCT chow consumption per cage was measured every week for 10 weeks, demonstrating no difference between the two groups (5 cages for each condition, 3 mice in each cage). FIG. 1E illustrates stool TG was normalized to the stool dry weight (N=3). FIG. 1F illustrates photographs of mice from each treatment group, demonstrating reduction of liver size and increased redness with NCT, as well as visceral adiposity. Red box indicates liver and arrow indicates epididymal fat pad. FIG. 1G photographs of dissected livers demonstrating reduction of liver size and increased redness with NCT. FIG. 1H illustrates liver weight was normalized to the body weight (NC, N=5, HFD and HFD+NCT, N=15). FIG. 1I illustrates photomicrographs of liver sections stained with Oil red O. FIG. 1J illustrates quantification of Oil red O staining HFD and HFD+NCT values were measured using image J with consistent threshold settings and normalized to NC values to calculate fold change (NC, N=5, HFD and HFD+NCT, N=15). FIG. 1K illustrates hepatic TG level was normalized to the liver weight (NC, N=5, HFD and HFD+NCT, N=15). FIG. 1L illustrates representative pictures of epididymal fat pads from each group. FIG. 1M illustrates epididymal fat pad weight was quantified by normalizing with total mouse body weight (NC, N=5, HFD and HFD+NCT, N=15). FIG. 1N illustrates photographs of mice from each treatment group, demonstrating reduction of subcutaneous fat (arrow indicated). Dots indicate individual mice. Values represent the mean±SEM. *p<0.05, **p<0.01, ***p<0.001 (HFD vs NC or HFD+NCT). NS=non-significant. Scale bar=200 μM.

FIG. 2A illustrates FAO activity in liver lysate prepared in the presence of octanoyl-CoA. FIG. 2B illustrates FAO activity prepared in the absence of octanoyl-CoA. FIG. 2C illustrates FAO activity+octanoyl CoA minus FAO activity −octanoyl CoA. FIG. 2D illustrates total hepatic NAD level. Dots indicate individual mice (N=6). Values represent the mean SEM. *p<0.05, **p<0.01 (HFD vs HFD+NCT). NS=non-significant.

FIG. 3A illustrates Liver sections from NC, HFD, and HFD+NCT mice were immunostained for VDAC-1 (red color). DAPI (blue) is for nuclear staining. FIG. 3B illustrates Quantification of hepatic VDAC-1 staining intensity (fold change vs NC, NC, N=3, HFD and HFD+NCT, N=12). FIG. 3C illustrates Citrate synthase (CS) activity in liver lysate was reduced by HFD, which was reversed by NCT (NC, N=3, HFD and HFD+NCT, N=12). FIG. 3D illustrates Western blots for cytochrome C and SDHA. After detecting each protein, the membrane was stained for Ponceau S as a control for protein loading. FIGS. 3E-F illustrate Quantification of cytochrome C and SDHA protein expression, respectively, in mouse liver. Each value was first normalized to the Ponceau S staining and the fold change was then calculated vs NC (N=6). FIGS. 3G-H illustrate qPCR analysis in mouse liver for cytochrome C and SDHA mRNA expression normalized with 18s rRNA (NC, N=3, HFD and HFD+NCT, N=12). FIGS. 3I-J illustrate qPCR analysis of cytochrome C and SDHA mRNA from human primary hepatocytes cultured in the indicated concentration of NCT (0, 5, 15, 40 μM). Values were normalized with 18s rRNA (N=3). FIGS. 3K-L illustrate qPCR analysis in mouse liver for mitochondrial DNA (ND1, 16s) expression normalized with HK2 (N=4). FIG. 3M illustrates qPCR analysis in mouse liver for HSP60 mRNA expression normalized with 18s rRNA (NC, N=3, HFD and HFD+NCT, N=12). FIG. 3N illustrates qPCR analysis in mouse liver for PPARγ mRNA expression normalized with 18s rRNA (NC, N=3, HFD and HFD+NCT, N=12). Each dot indicates an individual mouse or human donor. Values represent the mean±SEM. *p<0.05, **p<0.01, ***p<0.001 (HFD v 623 s NC or HFD+NCT, 0 μM vs each concentration of NCT in human hepatocyte). NS=non-significant. Scale bar=200 μM

FIG. 4A illustrates Representative pictures of western blot analysis for PPARGC1A expression and Ponceau S in mouse liver. FIG. 4B illustrates PPARGC1A protein expression (fold change vs NC) in mouse liver was normalized with Ponceau S (N=6). FIGS. 4C-E illustrate qPCR analysis in mouse liver of PPARGC1A, Sirtuin1, Sirtuin3 mRNA level normalized with 18s rRNA (NC, N=3, HFD and HFD+NCT, N=12). FIGS. 4F-H illustrates qPCR analysis of PPARGC1A, Sirtuin1, and Sirtuin3 mRNA level normalized with 18s rRNA in human primary hepatocytes (N=3). Dots indicate individual mouse or human donors. Values represent the mean±SEM. *p<0.05, **p<0.01 (HFD vs NC or HFD+NCT, 0 μM vs each concentration of NCT in human hepatocyte). NS=non-significant.

FIGS. 5A-C illustrate qPCR analysis in mouse liver of IL-6, TNFα, and IL-1P mRNA levels normalized to 18s rRNA (NC, N=3, HFD and HFD+NCT, N=12). FIG. 5D illustrates ELISA analysis for IL-6 secreted into the medium of human primary hepatocytes cultured in palmitate and DMSO (vehicle control) or different concentrations of NCT (N=3). FIGS. 5E-F illustrate qPCR analysis of IL-6 and TNFα mRNA level in primary human hepatocytes. Normalization was to 18s rRNA (N=3). FIGS. 5G-H illustrate Nitric oxide (NO) analysis in in vivo and in vitro. NO expression normalized to liver weight in vivo (NC, N=3; HFD and HFD+NCT, N=4) and normalized to protein in T6PNE cells in vitro (N=3). FIG. 5I illustrates Alanine aminotransferase (ALT) level in blood was reduced in HFD+NCT mice compared to HFD mice (NC, N=3; HFD, N=12; HFD+NCT, N=9). Dots indicate individual mouse or human donors. Values represent the mean±SEM. *p<0.05, **p<0.01, ***p<0.001 (HFD vs NC or HFD+NCT, 0 μM vs each concentration of NCT in human hepatocyte and T6PNE cells). NS=non-significant.

FIG. 6A illustrates precipitation of NCT on subcutaneous injection site on representative mouse. Red arrow indicates the compound precipitation. FIG. 6B illustrates representative picture of dissected subcutaneous NCT and DMSO injected mouse. FIG. 6C illustrates dissected liver pictures of representative mice (N=6). No difference in liver color. FIG. 6D illustrates liver weight (normalized with body weight) was measured (N=6). FIG. 6E illustrates representative picture of dissected methyl cellulose and NCT treated mice (N=5). FIG. 6F illustrates representative picture of dissected stomach of methyl cellulose (MC) and NCT treated mice (N=5). Right: intact stomach Left: opened stomach and red arrows indicates the NCT precipitation inside stomach. FIG. 6G illustrates representative picture of dissected liver. No difference in liver color. FIG. 6H illustrates representative pictures of Oil Red O staining on liver sections. FIG. 6I illustrates NCT compound level measured in serum and stool (N=4). Dots indicate individual mice, NS=non-significant. Values represent the mean±SEM. Scale bar=200 μM.

FIG. 7A illustrates Immunostaining of cleaved caspase3 (red) and DAPI (blue). FIG. 7B illustrates Total DAPI number per well. FIG. 7C illustrates Quantification of cleaved caspase3 positive cells normalized to cell number measured with DAPI. FIG. 7D illustrates Immunostaining of cleaved caspase3 (red), HNF4α (green) and DAPI (blue) in mouse liver. Values represent the mean±SEM. NS=non-significant. Scale bar=200 μM.

FIG. 8A illustrates TG level in serum samples collected from mice after 10 weeks of HFD and NCT treated HFD chow fed mice was quantified (N=6). FIG. 8B illustrates free fatty acid in serum sample of these mice was quantified (N=15). FIG. 8C illustrates ALP level in the blood samples of these mice was quantified (N=9). Dots indicate individual mice, NS=non-significant. Values represent the mean±SEM.

FIGS. 9A-9B illustrates qPCR analysis in mouse liver of Parp1 and Parp2 mRNA expression normalized with 18s (NC, N=3, HFD and NCT, N=12). Dots indicate individual mice. Values represent the mean±SEM. NS=non-significant.

FIG. 10 illustrates a table describing NCT dose and duration of chow treatment and post treatment monitoring.

FIG. 11 illustrates a table of a liver profile panel and hematological analysis.

FIG. 12 illustrates a table of a liver profile panel and hematological analysis.

### DETAILED DESCRIPTION

This disclosure provides, among other things, the discovery of strong HNF4α agonists and their use to uncover a previously unknown pathway by which HNF4α controls the level of fat storage in the liver. While not wishing to be bound by theory, it is believed that this involves the induction of lipophagy by dihydroceramides, the synthesis and secretion of which is controlled by genes induced by HNF4α. The HNF4α activators are N-transcaffeoyltyramine (NCT) and N-transferuloyltyramine (NFT), which are structurally related to known drugs alverine and benfluorex, which are weak HNF4α activators. With in vitro studies described herein, NCT and NFT induced fat clearance from palmitate-loaded cells.

In aspects, compounds and compositions containing tyramine containing hydroxycinnamic acid amides are provided herein. Some embodiments provided herein provide for the compounds and compositions for the use in methods of promoting increased mitochondrial mass and function.

### Compositions

In some aspects, the disclosure provided herein disclosure provides plant-derived aromatic metabolites with one or more acidic hydroxyl groups attached to aromatic arenes, and their use in modulating metabolism. In one embodiment, the plant-derived aromatic metabolite is a structural analog of compound 1:

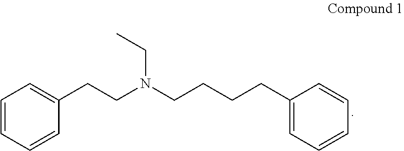 [SMILES: CCN(CCCCc1ccccc1)CCc1ccccc1]

In particular, the disclosure encompasses a compound of Formula (I), or an isomer, salt, homodimer, heterodimer, or conjugate thereof:

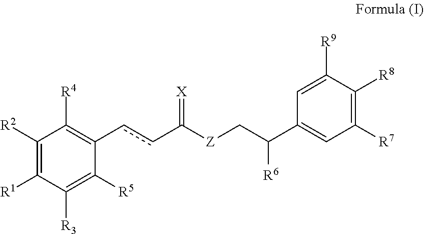

In some embodiments, R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R⁴, R⁵, R⁶, R⁷, and R⁹ are each independently hydrogen, deuterium, hydroxyl, or halogen;

In some embodiments, R¹, R², and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R³, R⁴, R⁵, R⁶, R⁷, and R⁹ are each independently hydrogen, deuterium, hydroxyl, or halogen.

In some embodiments, the dashed bond is present or absent.

In some embodiments, X is CH₂ or O.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (I) is provided as a pharmaceutically acceptable salt or solvate thereof.

In some embodiments, a compound of Formula (I) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

In some embodiments, the disclosure encloses a compound of Formula (II):

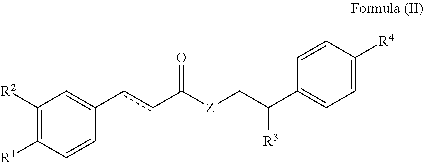

In some embodiments, R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, the dashed bond is present or absent.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (II) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

In some embodiments, a compound of Formula (II) is provided as a pharmaceutically acceptable salt or solvate thereof.

In some embodiments, the disclosure encloses a compound of Formula (III):

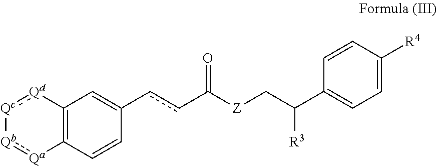

In some embodiments, R³ and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆ alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₂₋₁₂heterocyclyl, optionally substituted —(O)C₅₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, the each independently selected dashed bond is present or absent.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted —(O)C₄₋₁₂cycloalkyl, optionally substituted —(O)C₄₋₁₂heterocyclyl, optionally substituted —(O)C₄₋₁₂cycloalkyl, optionally substituted —(O)C₁₋₆alkylC₅₋₁₂aryl, optionally substituted —(O)C₁₋₆alkylC₅₋₁₂heteroaryl.

In some embodiments, Q^{a} Q^{b}, Q^{c}, Q^{d} are each independently selected from a bond, CHR^{a}, NR^{a}, C=O, and —O—.

In some embodiments, R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, Q^{c}, Q^{d} are absent. In some embodiments, Q^{d} is absent.

In some embodiments, n is 1, 2, 3, or 4

In some embodiments, a compound of Formula (II) is provided as a pharmaceutically acceptable salt or solvate thereof.

“Isomer” refers to especially optical isomers (for example essentially pure enantiomers, essentially pure diastereomers, and mixtures thereof) as well as conformation isomers (i.e., isomers that differ only in their angles of at least one chemical bond), position isomers (particularly tautomers), and geometric isomers (e.g., cis-trans isomers).

In certain embodiments, a compound of Formula (I) or Formula (II) is selected from:

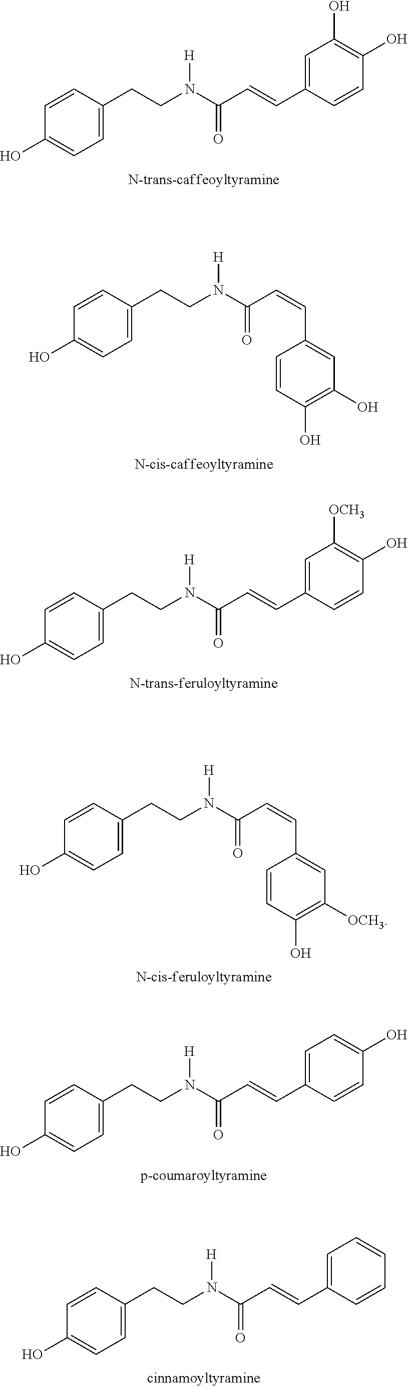 [SMILES: O=C(/C=C/c1ccc(O)c(O)c1)NCCc1ccc(O)cc1 ; O=C(/C=C\c1ccc(O)c(O)c1)NCCc1ccc(O)cc1 ; COc1cc(/C=C/C(=O)NCCc2ccc(O)cc2)ccc1O ; COc1cc(/C=C\C(=O)NCCc2ccc(O)cc2)ccc1O ; O=C(/C=C/c1ccc(O)cc1)NCCc1ccc(O)cc1 ; O=C(/C=C/c1ccccc1)NCCc1ccc(O)cc1]

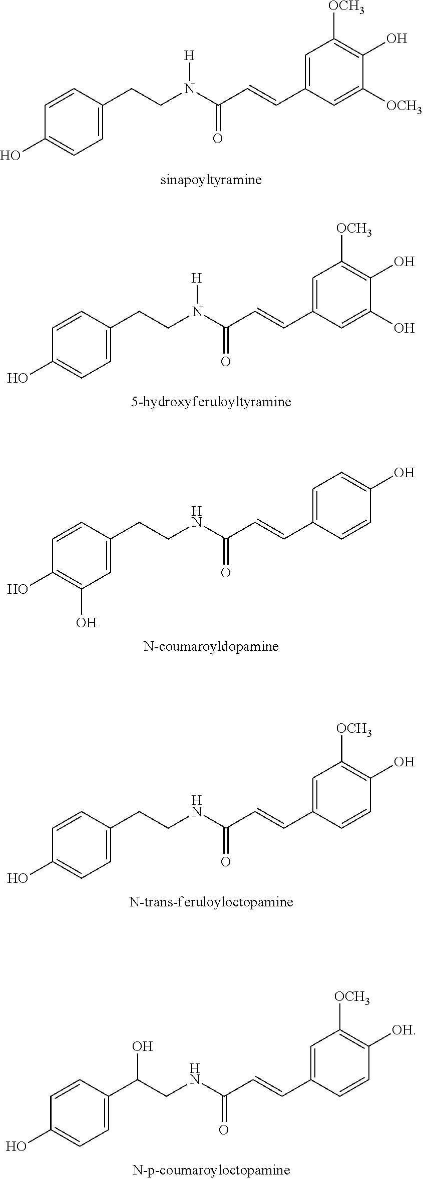 [SMILES: COc1cc(/C=C/C(=O)NCCc2ccc(O)cc2)cc(OC)c1O ; COc1cc(/C=C/C(=O)NCCc2ccc(O)cc2)cc(O)c1O ; O=C(/C=C/c1ccc(O)cc1)NCCc1ccc(O)c(O)c1 ; COc1cc(/C=C/C(=O)NCCc2ccc(O)cc2)ccc1O ; COc1cc(/C=C/C(=O)NCC(O)c2ccc(O)cc2)ccc1O]

A salt of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like; or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic camphorsulfonic acid, acid, 4-toluenesulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

As is known in the art, a homodimer is a molecule composed of two identical tyramine containing hydroxycinnamic acid amide subunits. By comparison, a heterodimer is a molecule composed of two different tyramine containing hydroxycinnamic acid amide subunits. Examples of homodimers of this disclosure include but are not limited to a cross-linked N-trans-feruloyltyramine dimer, a cross-linked N-trans-caffeoyl tyramine dimer and a cross-linked p-coumaroyltyramine dimer. See, for example, King & Calhoun (2005) Phytochemistry 66(20): 2468-73, which teaches the isolation of a cross-linked N-transferuloyltyramine dimer from potato common scab lesions.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, mono-substituted amino group and di-substituted amino group, and protected derivatives thereof.

For the groups herein, the following parenthetical subscripts further define the groups as follows: “(Cₙ)” defines the exact number (n) of carbon atoms in the group. For example, “C₁-C₆-alkyl” designates those alkyl groups having from 1 to 6 carbon atoms (e.g., 1, 2, 3, 4, 5, or 6, or any range derivable therein (e.g., 3-6 carbon atoms)).

In addition to isomers, salts, homodimers, heterodimers, and conjugates, the tyramine containing hydroxycinnamic acid amide may also be glycosylated. A glycosylated tyramine containing hydroxycinnamic acid amide may be produced by transglycosylating the tyramine containing hydroxycinnamic acid amide to add glucose units, for example, one, two, three, four, five, or more than five glucose units, to the tyramine containing hydroxycinnamic acid amide. Transglycosylation can be carried out with any suitable enzyme including, but not limited to, a pullulanase and isomaltase (Lobov, et al. (1991) Agric. Biol. Chem. 55:2959-2965), ˜-galactosidase (Kitahata, et al. (1989) Agric. Biol. Chem. 53:2923-2928), dextrine saccharase (Yamamoto, et al. (1994) Biosci. Biotech. Biochem. 58: 1657-1661) or cyclodextrin gluconotransferase, with pullulan, maltose, lactose, partially hydrolyzed starch and maltodextrin being donors.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyls. The alkyl group may be substituted or unsubstituted.

The term “halogen atom” or “halogen” as used herein, means any one of the radio-stable atoms of column 7 of the Periodic Table of the Elements, such as chloro (Cl), fluoro (F), bromo (Br) and iodo (I) groups.

In any of the groups described herein, an available hydrogen may be replaced with an alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, alkoxycarbonyl, acyl, halo, nitro, aryloxycarbonyl, cyano, carboxy, aralkoxycarbonyl, alkylsulfonyl, arylsulfonyl, heteroarylsulfonyl, alkylthio, arylthio, heteroarylthio, aralkylthio, heteroaralkylthio, cycloalkyl, or heterocyclyl.

Any undefined valency on an atom of a structure shown in this application implicitly represents a hydrogen atom bonded to the atom.

As used herein, “alkenyl” refers to an alkyl group, as defined herein, that contains in the straight or branched hydrocarbon chain one or more double bonds. An alkenyl group may be unsubstituted or substituted.

As used herein, “alkynyl” refers to an alkyl group as defined herein, that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl, and cyclooctyl.

As used herein, “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene, and azulene. An aryl group may be substituted or unsubstituted.

As used herein, “heterocyclyl” refers to mono- or polycyclic ring systems including at least one heteroatom (e.g., O, N, S). Such systems can be unsaturated, can include some unsaturation, or can contain some aromatic portion, or be all aromatic. A heterocyclyl group can contain from 3 to 30 atoms. A heterocyclyl group may be unsubstituted or substituted.

In particular embodiments, R¹ is present and represents a hydroxy group at the para position and R² is a hydroxy or lower alkoxy group at the meta position. In certain embodiments, the tyramine containing hydroxycinnamic acid amide having the structure of Formula (I) is in the trans configuration.

As used herein, “heteroaryl” refers to a monocyclic or multicyclic aromatic ring system (a ring system having a least one ring with a fully delocalized pi-electron system) that contain(s) one or more heteroatoms, that is, an element other than carbon, including but not limited to, nitrogen, oxygen, and sulfur, and at least one aromatic ring. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

The term “amino” as used herein refers to a —NH₂ group.

As used herein, the term “hydroxy” refers to a —OH group.

A “cyano” group refers to a “—CN” group.

A “carbonyl” group refers to a C═O group.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. An N-amido may be substituted or unsubstituted.

A “urea” group refers to a “—N(R_{A}R_{B})—C(═O)—N(R_{A}R_{B})—” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A urea group may be substituted or unsubstituted.

The term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic acid, acetic acid (AcOH), propionic acid, glycolic acid, pyruvic acid, malonic acid, maleic acid, fumaric acid, trifluoroacetic acid (TFA), benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Pharmaceutical salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns.

Likewise, it is understood that, in any compound described, all tautomeric forms are also intended to be included.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the methods and formulations described herein include the use of crystalline forms, amorphous phases, and/or pharmaceutically acceptable salts, solvates, hydrates, and conformers of compounds of some embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. In specific embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein. Other forms in which the compounds of some embodiments can be provided include amorphous forms, milled forms and nano-particulate forms.

Likewise, it is understood that the compounds described herein, such as compounds of some embodiments, include the compound in any of the forms described herein (e.g., pharmaceutically acceptable salts, prodrugs, crystalline forms, amorphous form, solvated forms, enantiomeric forms, tautomeric forms, and the like).

### Formulations

A substantially pure compound or extract comprising a compound of this disclosure can be combined with a carrier and provided in any suitable form for consumption by or administration to a subject. In this respect, the compound or extract is added as an exogenous ingredient or additive to the consumable. Suitable consumable forms include, but are not limited to, a dietary supplement, food ingredient or additive, a medical food, nutraceutical or pharmaceutical composition. In some embodiments, the compound or extract is provided in either a liquid or powder form.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound of Formula (I), (II), (III), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example., syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In some embodiments, a compound of Formula (I), Formula (II), or Formula (III) is administered at a dose in the range of about 0.1-200 mg/kg body weight. In some embodiments, a compound of Formula (I), Formula (II), or Formula (III) is administered at a dose in the range of about 0.1-1, 0.5-1, 0.1-10, 0.5-10, 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500, 60-600, 60-700, 60-800, 60-900, 60-1000, 70-80, 70-90, 70-100, 70-200, 70-300, 70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, a compound of Formula (I), Formula (II), or Formula (III) is administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 80, 90, or 95 mg/kg of the body weight. In some embodiments, a compound of Formula (I), Formula (II), or Formula (III) is administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, a compound of Formula (I), Formula (II), or Formula (III) is administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of a subjects body weight.

In some embodiments, a compound of Formula (I), Formula (II), or Formula (III) dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg 50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, a compound of Formula (I), Formula (II), or Formula (III) administered is about 20 mg-60 mg, 27 mg-60 mg, 20 mg-45 mg, or 27 mg-45 mg. In some embodiments, a compound of Formula (I), Formula (II), or Formula (III) administered is about 10 mg to about 120 mg. In some embodiments, a compound of Formula (I), Formula (II), or Formula (III) administered is about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, a compound of Formula (I), Formula (II), or Formula (III) dose is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg. about 400 mg, about 500 mg, about 600 mg, about 700 mg, about 800 mg, about 900 mg, or about 1000 mg. In some embodiments, a compound of Formula (I), Formula (II), or Formula (III) dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

The term “carrier” as used herein means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other nontoxic compatible substances employed in conventional formulations.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

In particular embodiments of this disclosure, a consumable composition includes the compound or extract, a carrier and a preservative to reduce or retard microbial growth. In some embodiments, the preservative is added in amounts up to about 5%. In some embodiments, the composition includes a preservative from about 0.01%, 0.1%, 1%, 2%, 3%, 4%, 5%, or ranges including and/or spanning the aforementioned values. In some embodiments, the preservative is from about 0.01% to about 1% by weight of the composition. In some embodiments, the preservative is from about 1% to about 5% by weight of the composition. Examples of preservatives include, but are not limited to, sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA).

The liquid forms in which the compound or extract of the disclosure is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

When a compound or extract of the present disclosure is administered as pharmaceuticals, nutraceuticals, or dietary supplements to humans and animals, they can be given per se or as a composition containing, for example, 0.1 to 99% active ingredient in combination with an acceptable carrier. In some embodiments, the compound or extract of the present disclosure may be administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values.

A consumable product may be consumed by a subject to provide less than 100 mg of a compound disclosed herein per day. In certain embodiments, the consumable provides between 10 and 60 mg/day of a tyramine containing hydroxycinnamic acid amide. In certain embodiments, the consumable provides between 10 and 120 mg/day of a tyramine containing hydroxycinnamic acid amide. In certain embodiments, the consumable provides between 100 and 200 mg/day of a tyramine containing hydroxycinnamic acid amide. The effective amount can be established by methods known in the art and be dependent upon bioavailability, toxicity, etc.

While it is contemplated that individual tyramine containing hydroxycinnamic acid amides may be used in the consumables of this disclosure, it is further contemplated that two or more of the compounds or extracts could be combined in any relative amounts to produce custom combinations of ingredients containing two or more tyramine containing hydroxycinnamic acid amides in desired ratios to enhance product efficacy, improve organoleptic properties or some other measure of quality important to the ultimate use of the product.

### Combinations

Some aspects relate to a combination of a compound of Formula (I), (II), or (III) with one or more compounds selected from a dihydrosphingosine, ceramide, glycosphingolipid, and a sphingosine. In some embodiments, the combination includes more compounds selected from dihydroceramide, ceramide, or a sphingosine.

In some embodiments, the ceramide is selected from the group consisting of natural ceramide, synthetic ceramide, a ceramide phosphate, a 1-O-acyl-ceramide, a dihydroceramide, a dihydroceramide phosphate, and a 2-hydroxy ceramide.

In some embodiments, the natural ceramide is porcine brain or egg.

In some embodiments, he synthetic ceramide is selected from the group consisting of N-octadecanoyl-D-erythro-sphingosine (C18), N-hexadecanoyl-D-erythro-sphingosine (C16)N-acetoyl-D-erythro-sphingosine (C2 Ceramide, d18:1/2:0), N-butyroyl-D-erythro-sphingosine (C4 Ceramide, d18:1/4:0), N-hexanoyl-D-erythro-sphingosine (C6 Ceramide, d18:1/6:0), N-octanoyl-D-erythro-sphingosine (C8 Ceramide, d18:1/8:0), N-decanoyl-D-erythro-sphingosine (C10 Ceramide, d18:1/10:0), N-lauroyl-D-erythro-sphingosine (C12 Ceramide, d18:1/12:0), N-myristoyl-D-erythro-sphingosine (C14 Ceramide, d18:1/14:0), N-palmitoyl-D-erythro-sphingosine (C16 Ceramide, d18:1/16:0), N-heptadecanoyl-D-erythro-sphingosine (C17 Ceramide, d18:1/17:0), N-stearoyl-D-erythro-sphingosine (C18 Ceramide, d18:1/18:0), N-oleoyl-D-erythro-sphingosine (C18:1 Ceramide, d18:1/18:1(9Z)), N-arachidoyl-D-erythro-sphingosine (C20 Ceramide, d18:1/20:0), N-behenoyl-D-erythro-sphingosine (C22 Ceramide, d18:1/22:0), N-lignoceroyl-D-erythro-sphingosine (C24 Ceramide, d18:1/24:0), N-nervonoyl-D-erythro-sphingosine (C24:1 Ceramide, d18:1/24:1(15Z)), N-acetoyl-D-erythro-sphingosine (C17 base) (C2 Ceramide, d17:1/2:0), N-octanoyl-D-erythro-sphingosine (C17 base) (C8 Ceramide, d17:1/8:0), N-stearoyl-D-erythro-sphingosine (C17 base) (C18 Ceramide, d17:1/18:0), N-oleoyl-D-erythro-sphingosine (C17 base) (C18:1 Ceramide, d17:1/18:1(9Z)), N-arachidoyl-D-erythro-sphingosine (C17 base) (C20 Ceramide, d17:1/20:0), N-lignoceroyl-D-erythro-sphingosine (C17 base) (C24 Ceramide, d17:1/24:0), and N-nervonoyl-D-erythro-sphingosine (C17 base) (C24:1 Ceramide, d17:1/24:1(15Z)).

In some embodiments, the ceramide phosphate is selected from the group consisting of N-acetoyl-ceramide-1-phosphate (ammonium salt) (C2 Ceramide-1-Phosphate, d18:1/2:0), N-octanoyl-ceramide-1-phosphate (ammonium salt) (C8 Ceramide-1-Phosphate, d18:1/8:0), N-lauroyl-ceramide-1-phosphate (ammonium salt) (C12 Ceramide-1-Phosphate, d18:1/12:0), N-palmitoyl-ceramide-1-phosphate (ammonium salt) (C16 Ceramide-1-Phosphate, d18:1/16:0), N-oleoyl-ceramide-1-phosphate (ammonium salt) (C18:1 Ceramide-1-Phosphate, d18:1/18:1(9Z)), N-lignoceroyl-ceramide-1-phosphate (ammonium salt) (C24 Ceramide-1-Phosphate, 18:1/24:0), N-acetoyl-ceramide-1-phosphate (C17 base) (ammonium salt) (C2 Ceramide-1-Phosphate, d17:1/2:0), and N-octanoyl-ceramide-1-phosphate (C17 base) (ammonium salt) (C8 Ceramide-1-Phosphate, d17:1/8:0).

In some embodiments, the dihydroceramide is selected from the group consisting of N-hexanoyl-D-erythro-sphinganine (C6 Dihydroceramide, d18:0/6:0), N-octanoyl-D-erythro-sphinganine (C8 Dihydroceramide, d18:0/8:0), N-palmitoyl-D-erythro-sphinganine (C16 Dihydroceramide, d18:0/16:0), N-stearoyl-D-erythro-sphinganine (C18 Dihydroceramide, d18:0/18:0), N-oleoyl-D-erythro-sphinganine (C18:1 Dihydroceramide, d18:0/18:1(9Z)), N-lignoceroyl-D-erythro-sphinganine (C24 Dihydroceramide, d18:0/24:0), and N-nervonoyl-D-erythro-sphinganine-D-erythro-sphinganine (C24:1 Dihydroceramide, d18:0/24:1(15Z)).

In some embodiments, the dihydroceramide phosphate is N-palmitoyl-D-erythro-dihydroceramide-1-phosphate (ammonium salt) (C16 Dihydroceramide-1-Phosphate, d18:0/16:0) or N-lignoceroyl-D-erythro-dihydroceramide-l-phosphate (ammonium salt) (C24 Dihydroceramide-1-Phosphate, d18:0/24:0).

In some embodiments, the 2-hydroxy ceramide is selected from the group consisting of N-(2′-(R)-hydroxylauroyl)-D-erythro-sphingosine (12:0(2R—OH) Ceramide), N-(2′-(S)-hydroxylauroyl)-D-erythro-sphingosine (12:0(2S—OH) Ceramide), N-(2′-(R)-hydroxypalmitoyl)-D-erythro-sphingosine (16:0(2R—OH) Ceramide), N-(2′-(S)-hydroxypalmitoyl)-D-erythro-sphingosine (16:0(2S—OH) Ceramide), N-(2′-(R)-hydroxyheptadecanoyl)-D-erythro-sphingosine (17:0(2R—OH) Ceramide), N-(2′-(S)-hydroxyheptadecanoyl)-D-erythro-sphingosine (17:0(2S—OH) Ceramide), N-(2′-(R)-hydroxystearoyl)-D-erythro-sphingosine (18:0(2R—OH) Ceramide), N-(2′-(S)-hydroxystearoyl)-D-erythro-sphingosine (18:0(2S—OH) Ceramide), N-(2′-(R)-hydroxyoleoyl)-D-erythro-sphingosine (18:1(2R—OH) Ceramide), N-(2′-(S)-hydroxyoleoyl)-D-erythro-sphingosine (18:1(2S—OH) Ceramide), N-(2′-(R)-hydroxyarachidoyl)-D-erythro-sphingosine (20:0(2R OH) Ceramide), N-(2′-(S)-hydroxylarachidoyl)-D-erythro-sphingosine (20:0(2S—OH) Ceramide), N-(2′-(R)-hydroxybehenoyl)-D-erythro-sphingosine (22:0(2R—OH) Ceramide), N-(2′-(S)-hydroxylbehenoyl)-D-erythro-sphingosine (22:0(2S—OH) Ceramide), N-(2′-(R)-hydroxylignoceroyl)-D-erythro-sphingosine (24:0(2R—OH) Ceramide), N-(2′-(S)-hydroxyllignoceroyl)-D-erythro-sphingosine (24:0(2S—OH) Ceramide), N-(2′-(R)-hydroxynervonoyl)-D-erythro-sphingosine (24:1(2R—OH) Ceramide), and N-(2′-(S)-hydroxylnervonoyl)-D-erythro-sphingosine (24:1(2S—OH) Ceramide).

In some embodiments, the sphingosine is selected from the group consisting of natural sphingosine, synthetic sphingosine, phosphorylated sphingosine (S1P), and methylated sphingosine.

In some embodiments, the natural sphingosine is D-erythro-sphingosine.

In some embodiments, the synthetic sphingosine is selected from the group consisting of sphingosine (d18:1), sphingosine (d17:1), sphingosine (d20:1), L-threo-sphingosine (d18:1), 1-deoxysphingosine, and 1-desoxymethylsphingosine. In some embodiments, the sphinganine is selected from the group consisting of sphinganine (d18:0), sphinganine (d17:0), sphinganine (d20:0), 1-deoxysphinganine, 1-desoxymethylsphinganine, and L-threo-dihydrosphingosine (d18:0) (Safingol). In some embodiments, the phosphorylated sphingosine is selected from the group consisting of sphingosine-1-phosphate (d18:1), sphingosine-1-phosphate (DMA Adduct), sphingosine-1-phosphate (d17:1), sphingosine-1-phosphate (d20:1), sphinganine-1-phosphate (d18:0), sphinganine-1-phosphate (d17:0), and sphinganine-1-phosphate (d20:0). In some embodiments, the methylated sphingosine is selected from the group consisting of monomethyl sphingosine (d18:1), dimethyl sphingosine (d18:1), dimethyl sphingosine (d17:1), trimethyl sphingosine (d18:1), trimethyl sphingosine (d17:1), dimethyl sphinganine (d18:0), trimethyl sphinganine (d18:0), dimethyl sphingosine-1-phosphate (d18:1), and dimethyl sphinganine-1-phosphate (d18:0).

In some embodiments, the glycosphingolipid is selected from the group consisting of a natural glycosphingolipid, a glycosyl sphingolipid, a galactosyl sphingolipid, a lactosyl sphingolipid, a sulfatide, and a-galactosyl ceramide (αGalCer).

In some embodiments, the natural glycosphingolipid is selected from the group consisting of a cerebroside (e.g., from porcine brain), a glucocerebroside (e.g., from soy), a sulfatide (ammonium salt) (e.g., from porcine brain), a GM1 ganglioside (ammonium salt) (e.g., from ovine brain), a ganglioside GM1 (e.g., from ovine brain), and a total ganglioside extract (ammonium salt) (e.g., from porcine brain).

In some embodiments, the glycosyl sphingolipid is selected from the group consisting of D-glucosyl-β1-1′-D-erythro-sphingosine (Glucosyl(β) Sphingosine, d18:1), D-glucosyl-β-1,1′ N-octanoyl-D-erythro-sphingosine (C8 Glucosyl(β) Ceramide, d18:1/8:0), D-glucosyl-β-1,1′ N-lauroyl-D-erythro-sphingosine (C12 Glucosyl(β) Ceramide, d18:1/12:0), D-glucosyl-β-1,1′ N-palmitoyl-D-erythro-sphingosine (C16 Glucosyl(β) Ceramide, d18:1/16:0), D-glucosyl-β-1,1′ N-stearoyl-D-erythro-sphingosine (C18 Glucosyl(β) Ceramide, d18:1/18:0), D-glucosyl-β-1,1′ N-oleoyl-D-erythro-sphingosine (C18:1 Glucosyl(β) Ceramide, d18:1/18:1(9Z)), and D-glucosyl-β1-1′-N-nervonoyl-D-erythro-sphingosine (C24:1 Glucosyl(β) Ceramide, d18:1/24:1(15Z)).

In some embodiments, the galactosyl sphingolipid is selected from the group consisting of D-galactosyl-β1-1′-D-erythro-sphingosine (Galactosyl(p) Sphingosine, d18:1), N,N-dimethyl-D-galactosyl-β1-1′-D-erythro-sphingosine (Galactosyl(p) Dimethyl Sphingosine, d18:1), D-galactosyl-β-1,1′ N-octanoyl-D-erythro-sphingosine (C8 Galactosyl(p) Ceramide, d18:1/8:0), D-galactosyl-β-1,1′ N-lauroyl-D-erythro-sphingosine (C12 Galactosyl(p) Ceramide, d18:1/12:0), D-galactosyl-β-1,1′ N-palmitoyl-D-erythro-sphingosine (C16 Galactosyl(p) Ceramide, d18:1/16:0), and D-galactosyl-β-1,1′ N-nervonoyl-D-erythro-sphingosine (C24:1 Galactosyl(p) Ceramide, d18:1/24:1(15Z)).

In some embodiments, the lactosyl sphingolipid is selected from the group consisting of D-lactosyl-β1-1′-D-erythro-sphingosine (Lactosyl(β) Sphingosine, d18:1), D-lactosyl-β-1,1′ N-octanoyl-D-erythro-sphingosine (C8 Lactosyl(β) Ceramide, d18:1/8:0), D-lactosyl-β1-1′-N-octanoyl-L-threo-sphingosine (C8 L-threo-Lactosyl(β) Ceramide, d18:1/8:0), D-lactosyl-β-1,1′ N-lauroyl-D-erythro-sphingosine (C12 Lactosyl(β) Ceramide, d18:1/12:0), D-lactosyl-β-1,1′ N-palmitoyl-D-erythro-sphingosine (C16 Lactosyl(β) Ceramide, d18:1/16:0), D-lactosyl-β-1,1′ N-lignoceroyl-D-erythro-sphingosine (C24 Lactosyl(β) Ceramide, d18:1/24:0), and D-lactosyl-β1-1′-N-nervonoyl-D-erythro-sphingosine (C24:1 Lactosyl(β) Ceramide, d18:1/24:1).

In some embodiments, the sulfatide is selected from the group consisting of 3-O-sulfo-D-galactosyl-β1-1′-N-lignoceroyl-D-erythro-sphingosine (ammonium salt) (e.g., from porcine brain), 3-O-sulfo-D-galactosyl-β1-1′-N-lauroyl-D-erythro-sphingosine (ammonium salt) (C12 Mono-Sulfo Galactosyl(p) Ceramide, d18:1/12:0), 3-O-sulfo-D-galactosyl-β1-1′-N-heptadecanoyl-D-erythro-sphingosine (ammonium salt) (C17 Mono-Sulfo Galactosyl(p) Ceramide, d18:1/17:0), 3-O-sulfo-D-galactosyl-β1-1′-N-lignoceroyl-D-erythro-sphingosine (ammonium salt) (C24 Mono-Sulfo Galactosyl(p) Ceramide (d18:1/24:0), 3-O-sulfo-D-galactosyl-β1-1′-N-nervonoyl-D-erythro-sphingosine (ammonium salt) (C24:1 Mono-Sulfo Galactosyl(β) Ceramide, d18:1/24:1), and 3,6-di-O-sulfo-D-galactosyl-β1-1′-N-lauroyl-D-erythro-sphingosine (ammonium salt) (C12 Di-Sulfo Galactosyl(β) Ceramide, d18:1/12:0).

In some embodiments, the phosphospingolipid is selected from the group consisting of D-erythro-sphingosyl phosphoethanolamine (Sphingosyl PE, d18:1), N-lauroyl-D-erythro-sphingosyl phosphoethanolamine (C17 base) (C12 Sphingosyl PE, d17:1/12:0), and D-erythro-sphingosyl phosphoinositol (Sphingosyl PI).

In some embodiments, the phytosphingosine is selected from the group consisting of 4-hydroxysphinganine (Saccharomyces Cerevisiae) (D-ribo-Phytosphingosine), 4-hydroxysphinganine (C17 base) (D-ribo-phytosphingosine, C17 base), 4-hydroxysphinganine-N,N-dimethyl (Saccharomyces Cerevisiae) (Phytosphingosine-N,N-Dimethyl), 4-hydroxysphinganine-N,N,N-trimethyl (methyl sulfate salt) (Saccharomyces cerevisiae) (Phytosphingosine-N,N,N-Trimethyl), 4-hydroxysphinganine-1-phosphate (Saccharomyces Cerevisiae) (D-ribo-Phytosphingosine-1-Phosphate), 4-hydroxysphinganine-N,N-dimethyl-1-phosphate (ammonium salt) (Saccharomyces Cerevisiae) (Phytosphingosine-N,N-Dimethyl-1-Phosphate), N-acetoyl 4-hydroxysphinganine (Saccharomyces Cerevisiae) (N-02:0 Phytosphingosine), N-octanoyl 4-hydroxysphinganine (Saccharomyces Cerevisiae) (N-08:0 Phytosphingosine), N-stearoyl 4-hydroxysphinganine (Saccharomyces Cerevisiae) (N-18:0 Phytosphingosine), and 4-hydroxysphinganine-1-phosphocholine (Saccharomyces Cerevisiae) (Phytosphingosine Phosphocholine).

Some aspects relate to a combination of a compound of Formula (I), (II), or (III) with one or more compounds selected a macrolide, a retinide, and a DES1 inhibitor. In some embodiments, the one or more retinide is fenretinide, N-(4-hydroxyphenyl) retinamide (4-HPR), 4-oxo-N-(4-hydroxyphenyl) retinamide (4-oxo-HPR), or motretinide. In some embodiments, the DES1 inhibitor is selected from N-[(1R,2S)-2-hydroxy-1-hydroxymethyl-2-(2-tridecyl-1-cyclopropenyl)ethyl]octanamide (GT011) and (Z)-4-((5-(4-chlorophenyl)-1,3,4-oxadiazol-2-yl)amino)-N′-hydroxybenzimidamide (B-0027). In some embodiments, the one or more macrolide is selected from the group consisting of rapamycin, erythromycin, clarithromycin, roxithromycin, azithromycin, fidaxomicin, carbomycin A, josamycin, kitasamycin, midecamycin, oleandomycin, solithromycin, spiramycin, troleandomycin, tylosin, roxithromycin, telithromycin, cethromycin, solithromycin, solithromycin, tacrolimus, pimecrolimus, sirolimus, ciclosporin, polyene antimycotics, and cruentaren.

### Methods of Use

This disclosure provides for promoting increase in mitochondrial mass by providing a consumable composition as described herein and at least one carrier. In accordance with such methods, an effective amount of a composition as described herein is provided to a subject in need thereof thereby enhancing mitochondrial mass and function in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, increases mitochondrial mass. In some embodiments, a composition comprising a compound of Formula (I), Formula (II), or Formula (III) treats or ameliorates a disease or condition associated with enhancing mitochondrial mass in a subject. In some embodiments, a composition comprising a compound of Formula (I), Formula (II), or Formula (III) treats or ameliorates a disease or condition associated with enhancing mitochondrial mass in a subject. In some embodiments, a composition comprising a compound of Formula (I), Formula (II), or Formula (III) treats or ameliorates a disease or condition associated with mitochondrial mass and function.

In an embodiment, administering a composition comprising a compound of Formula (I), Formula (II), or Formula (III), or a pharmaceutically acceptable salt thereof, treats or improves at least one factor associated with mitochondrial mass and function of a subject. In other aspects, a composition comprising a compound of Formula (I), Formula (II), or Formula (III), or a pharmaceutically acceptable salt thereof disclosed herein enhances mitochondrial mass and function of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In yet other aspects, a composition comprising Formula (I), Formula (II), or Formula (III), or a pharmaceutically acceptable salt thereof, improves enhances mitochondrial mass and function in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In some embodiments, an effective amount of a composition as described herein is provided to a subject in need thereof thereby increasing fatty acid oxidation in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, increases fatty acid oxidation in a subject. In some embodiments, a composition comprising a compound of Formula (I), Formula (II), or Formula (III) treats or ameliorates a disease or condition associated with increasing fatty acid oxidation in a subject. In some embodiments, a composition comprising a compound of Formula (I), Formula (II), or Formula (III) treats or ameliorates a disease or condition associated with increasing fatty acid oxidation in a subject. In some embodiments, a composition comprising a compound of Formula (I), Formula (II), or Formula (III) treats or ameliorates a disease or condition associated with increasing fatty acid oxidation.

In an embodiment, administering a composition comprising a compound of Formula (I), Formula (II), or Formula (III), or a pharmaceutically acceptable salt thereof, treats or improves at least one factor associated with increasing fatty acid oxidation in a subject. In other aspects, a composition comprising a compound of Formula (I), Formula (II), or Formula (III), or a pharmaceutically acceptable salt thereof disclosed herein increasing fatty acid oxidation of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In yet other aspects, a composition comprising Formula (I), Formula (II), or Formula (III), or a pharmaceutically acceptable salt thereof, increasing fatty acid oxidation in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, administering a composition comprising a compound of Formula (I), Formula (II), or Formula (III), or a pharmaceutically acceptable salt thereof, treats or improves at least one factor associated with a high calorie diet in a subject. In other aspects, a composition comprising a compound of Formula (I), Formula (II), or Formula (III), or a pharmaceutically acceptable salt thereof disclosed herein treats fatty acid excess in a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In yet other aspects, a composition comprising Formula (I), Formula (II), or Formula (III), or a pharmaceutically acceptable salt thereof, reduces one or more signs of aging in a subject in need in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%. In yet other aspects, a composition comprising Formula (I), Formula (II), or Formula (III), or a pharmaceutically acceptable salt thereof, increases cellular energy in a subject in need in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In some embodiments, an effective amount of a composition as described herein is provided to a subject in need thereof thereby increasing NAD in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, increasing NAD in a subject. In some embodiments, a composition comprising a compound of Formula (I), Formula (II), or Formula (III) treats or ameliorates a disease or condition associated with increasing NAD in a subject. In some embodiments, a composition comprising a compound of Formula (I), Formula (II), or Formula (III) treats or ameliorates a disease or condition associated with increasing NAD in a subject. In some embodiments, a composition comprising a compound of Formula (I), Formula (II), or Formula (III) treats or ameliorates a disease or condition associated with increasing NAD.

In an embodiment, administering a composition comprising a compound of Formula (I), Formula (II), or Formula (III), or a pharmaceutically acceptable salt thereof, treats or improves at least one factor associated with increasing NAD in a subject. In other aspects, a composition comprising a compound of Formula (I), Formula (II), or Formula (III), or a pharmaceutically acceptable salt thereof disclosed herein increasing NAD of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values,

In some embodiments, an effective amount of a composition as described herein is provided to a subject in need thereof thereby inhibiting dihydroceramide conversion of ceramides in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, inhibiting dihydroceramide conversion of ceramides in a subject. In some embodiments, a composition comprising a compound of Formula (I), Formula (II), or Formula (III) treats or ameliorates a disease or condition associated ceramides in a subject. In some embodiments, a composition comprising a compound of Formula (I), Formula (II), or Formula (III) treats or ameliorates a disease or condition associated with ceramides in a subject. In some embodiments, a composition comprising a compound of Formula (I), Formula (II), or Formula (III) treats or ameliorates a disease or condition associated with ceramides in a subject.

In an embodiment, administering a composition comprising a compound of Formula (I), Formula (II), or Formula (III), or a pharmaceutically acceptable salt thereof, treats or improves at least one factor associated with inhibiting dihydroceramide conversion of ceramides in a subject. In other aspects, a composition comprising a compound of Formula (I), Formula (II), or Formula (III), or a pharmaceutically acceptable salt thereof disclosed herein inhibits dihydroceramide conversion of ceramides of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values.

In some embodiments, the subject is on a high fat diet. In some embodiments, the subject is on a high calorie diet. In some embodiments, the subject is obese. In some embodiments, the subject is obese prior to receiving the oral composition as described herein. In some embodiments, the subject is overweight. In some embodiments, the subject is overweight prior to receiving the oral composition as described herein.

In some embodiments, the subject is administered the oral composition for at least 1 day, 2 days, 3 days, 4 days, 5, days, 6, days, 1 week, 2, weeks, 3 weeks, 4 weeks, 5 weeks, 6 weeks, 7 weeks, 8 weeks, 3 months, 6 months, 1 year, or ranges including and/or spanning the aforementioned values. In some embodiments, the subject is administered the oral composition for at least 6 weeks. In some embodiments, the subject is administered the oral composition for at least 10 weeks.

In some embodiments, the subject's body weight is reduced by from about 1%, 2%, 3%, 4%, 5%, 6%, 7%, 8%, 9%, 10, 1%, 12%, 13%, 14%, 15%, 16%, 17%, 18%, 19%, 20%, 21%, 22%, 23%, 24%, 25%, or ranges including and/or spanning the aforementioned values. In some embodiments, the subject's body weight is reduced by from about 2% to about 20%.

In some aspects, the present disclosure provides a method of treating, preventing, ameliorating, or reducing muscle wasting or a muscle wasting disease in a subject suffering from such a disorder by administering an effective amount of a composition as described herein. The muscle wasting disease or conditions include, but is not limited to, the following: muscular dystrophies (such as DMD, BeckerMD, Limb-Girdle MD, Myotonic MD and FSHD), myositis (such as dermatomyositis, inclusion-body myositis, juvenile forms of myositis, polymyositis), myopathies (including inherited myopathy and acquired myopathy, such as diabetic myopathy or drug-induced myopathy), motoneuron diseases (such as Lou Gehrig's Disease or amyotrophic lateral sclerosis), myasthenia gravis, neurodegenerative diseases (such as Parkinson's disease, Huntington's disease and Alzheimer's disease), muscle wasting associated with cancers (such as pancreatic cancer, lung cancer, gastric cancer, ovarian cancer, colorectal cancer, melanomaleukemia, lung cancer, prostate cancer, brain cancer, bladder cancer, and head-neck cancer), muscle wasting associated with chronic heart failure (CHF), chronic kidney disease (CKD), liver failure, diabetes, chronic obstructive pulmonary disease (COPD), emphysema, cystic fibrosis, rheumatoid arthritis, osteoarthritis, liver fibrosis, cirrhosis, trauma (such as burns or motorcycle accident), bone fracture, organ transplantation (such as heart, lung, liver or kidney transplantation), ICU critical care, denervation (such as stoke or spinal cord injury), androgen deprivation therapy, corticosteroid therapy, infections (such as AIDS or tuberculosis), prolonged bed rest, sarcopenic obesity, and age-associated sarcopenia. In some embodiments, the muscle wasting is reduced in a subject by about, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In some embodiments, the subject is on a high fat diet.

In some aspects, the present disclosure provides a method of increasing or improving performance recovery in a subject in need thereof by administering an effective amount of a composition as described herein. In some embodiments, a performance recovery may include increased mobility in a subject. In some embodiments, a performance recovery may include increased endurance in a subject. In some embodiments, a performance recovery may include increased strength or power in a subject. In some embodiments, a performance recovery may include increased speed in a subject. In some embodiments, the performance is improved or increased in a subject by about, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In some embodiments, the subject is on a high fat diet.

### EXAMPLES

The following non-limiting examples are provided to further illustrate the present disclosure.

### Example 1

HNF4α is a nuclear receptor transcription factor that controls the expression of downstream genes that are important in multiple aspects of cellular metabolism. The classical view of HNF4α has been that its ligand binding pocket (LBP) is constitutively occupied by a fatty acid that plays a structural rather than regulatory role. However, it has been shown recently that the fatty acids in the HNF4α LBP are exchangeable in the context of full length HNF4α, particularly inside the cell. Using a cell-based promoter reporter assay for human insulin promoter activity that is highly sensitive to HNF4α activity, it was demonstrated that fatty acids act as HNF4α antagonists. Using the assay in high-throughput screening, it was discovered HNF4α antagonists and more recently agonists. Most recently, it was discovered a potent agonist, N-trans caffeoyltyramine. The availability of a potent HNF4α agonist facilitated in vivo studies that have revealed novel aspects of HNF4α, biology. Previously, it was shown that intraperitoneal (IP) administration of NCT reversed hepatic steatosis in obese mice fed a high fat diet through a previously unsuspected pathway involving regulation by HNF4α of dihydroceramide synthesis. Dihydroceramides were found to control hepatic lipophagy, leading to reversal of hepatic steatosis.

Here, it was uncovered another previously unsuspected effect of HNF4α that was made possible by the finding that it could be delivered orally mixed with high fat diet, allowing for longer term NCT administration. As with the previous studies with IP NCT administration, there was decreased hepatic steatosis. However, mice on high fat diet that were administered NCT for ten weeks had a much lower weight than mice on high fat diet alone. Decreased fat appeared to be due to an increase in fatty acid oxidation, which in turn was due to increased mitochondrial mass. Consistent with that, there was increased expression of mitochondrial proteins including VDAC1 and electron transport chain proteins, mitochondrial DNA, and total cellular NAD, most of which is in the mitochondria. There was a significant decrease in markers of inflammation and cellular stress, including nitric oxide. Thus, NCT is a strong candidate for a drug that can maintain metabolic homeostasis in the face of challenge from excess fatty acid intake.

### In Vivo Mouse Experiments

Four week-old male C57BL/6J (JAX cat #000664) mice were purchased from Jackson laboratory and were maintained in a 12-hour light/day cycle throughout the experiment. Prior to the experiments, mice were acclimated for two weeks. Six week old mice with similar body weights were randomized to normal diet (NC), high fat diet (HFD) (Research Diets, cat #D12492 60 kcal % fat) or HFD containing 4000 ppm NCT (HFD+NCT) (Research Diets, 60 kcal % fat+4000 ppm NCT), which was calculated to provide approximately 400 mg/kg/d NCT. HFD chow containing NCT was made with gray dye to distinguish it from regular HFD chow that had green dye. Mice for each treatment group were placed in separate cages. Equal amounts of fresh chow were provided every week to all mice. Body weight gain and food intake were measured every week for 10 weeks. After 10 weeks of chow treatment, mice were sacrificed for analysis. All animal experiments were approved by the Institutional Animal Care and Use Committee (IACUC) of the Sanford Burnham Prebys Medical Discovery Institute in accordance with national regulations. Sample size was chosen based on the prior study of NCT delivered by IP injection.

### Subcutaneous and Oral Gavage Treatment

12-week-old C57BL/6J DIO male mice were purchased from Jackson laboratory (cat #380050) and were fed with high fat diet (Research Diets, cat #D12492 60 kcal % fat). Prior to the experiments, mice were acclimated for two weeks. 14 week old mice were injected subcutaneously using sterile insulin syringes filled with DMSO or NCT (200 mg/kg of mouse body weight) bid for 2 weeks. For oral gavage, 14-week-old C57BL/6J DIO male mice were fed by oral gavage using a feeding needle attached to a sterile 1 ml syringe filled with 200 μL of methyl cellulose (MC, vehicle control) or 200 μL of NCT (200 mg/kg) dissolved in methyl cellulose twice a day for 2 weeks. All mice were maintained in a 12-hour light/day cycle throughout the experiment. For analysis all mice were sacrificed after 2 weeks of treatment.

### Sample Collection

Mouse samples were collected as described previously. Briefly, on the final day of treatment mice received dextrose (3 g/kg of body weight) by IP injection to stimulate insulin secretion, which inhibits FFA release from adipocytes, leaving liver-derived FFA as the major source of circulating FFA. One hour later, blood samples were collected via retro-orbital bleeding and mice were euthanized using pentobarbital. Mice were dissected aseptically and liver, epididymal fat, and body weights were measured and pictures taken. Dissected liver samples were washed immediately in sterile cold PBS and cut into small pieces. Half of the liver samples were snap frozen using liquid nitrogen and stored at −80° C. for RNA, protein isolation, and liver lysate preparations. The other half were fixed in 4% of cold paraformaldehyde (PFA, Santa Cruz Biotechnology, USA) and processed for histomorphometry and immunofluorescence.

### Oil Red O Staining (In Vivo) and Analysis:

Oil red O staining was performed as described previously (34). Slides containing frozen liver tissue sections from mice were air dried for 10-20 min followed by rehydration in distilled water. Sections were immersed in absolute propylene glycol (Cat #151957, MP Biomedicals, LLC, USA) for 2 min followed by 0.5% in Oil red O solution (Cat #K043, Poly Scientific R&D, USA) for 2 hours. Slides were then differentiated in 85% propylene glycol solution, washed with dH₂O for 2 hours, and mounted using glycerin jelly mounting medium. All slides were scanned at a magnification of 20× using the Aperio Scanscope FL system (Aperio Technologies Inc., Vista, CA, USA). The liver area stained with oil red O was measured using image J software as described, with some modifications as follow, Oil red O-stained liver images were opened in Image J software. Using the Analyze>Set Scale command, the scale bar of the images was set to 200 um. RGB images were then converted into gray scale images using the Image>Type>RGB Stack command and were split into red, blue and green channels. Using the Image >Adjust>Threshold command, the threshold was manually set to highlight the Oil red O stained lipid droplets in the green channel. It used the same threshold for all the images in all treatment groups and the % oil red O-stained area was obtained using the Analyze→Measure tool command. Fold change was calculated by normalizing the values to images from mice fed normal chow.

### Triglyceride Analysis

The TG level in mouse liver, serum and stool was measured according to manufacturer's instructions using the Triglyceride Calorimetric Assay Kit (Cat #10010303, Cayman Chemicals, USA). Liver and stool TG was normalized with liver and stool weight, respectively. Fold change was calculated by normalizing to values from mice fed NC.

### Free Fatty Acid Quantification

Blood samples were collected from dextrose injected mice and centrifuged to collect serum samples. The serum FFA level was measured according to manufacturer's instructions using a Free Fatty Acid Quantification Colorimetric/Fluorometric Kit (Cat #K612, BioVision, USA). Fold change was calculated by normalizing to values from mice fed NC.

### Immunofluorescence

Frozen liver sections were permeabilized using 0.3% Triton-X and incubated in antigen retrieval solution (Antigen retrieval citrate, Biogenex) at sub-boiling temperature for 10 min. Subsequently, sections were incubated with blocking buffer containing 5% normal donkey serum (Jackson Immuno Research) followed by incubation overnight at 4° C. with mouse anti-HNF4α monoclonal antibody (1:800, Cat #PP-H1415-00, R&D Systems), rabbit polyclonal anti-VDAC antibody (1:400, cat #PA1-954A, Invitrogen) and cleaved caspase3 antibody (1:500, cat #9664, Cell Signaling). Sections were washed and incubated for 1 hour at room temperature with anti-mouse secondary antibody coupled with Alexa fluor 488 (1:400, Invitrogen) and antirabbit secondary antibody coupled with rhodamine red. Nuclei were visualized by counterstaining with DAPI (40,6-diamidino-2-phenylindole, Sigma Aldrich). Slides were mounted using fluorescence mounting medium and images were obtained at 40× magnification using an Olympus IX71 fluorescence microscope. Fluorescence intensity of HNF4α-stained nuclei and VDAC stained mitochondria was calculated using MetaMorph TL software (version 7.6.5.0, Olympus). Fold change was calculated by normalizing to values from mice fed NC.

### Liver Profile Analysis

Mice were anesthetized and 100 μL of whole blood was collected via retro-orbital bleeding in lithium heparin blood collection tubes and transferred to single use VetScan mammalian liver profile reagent rotors. The levels of multiple analytes, including Alkaline Phosphatase (ALP), Alanine Aminotransferase (ALT), Gamma Glutamyl Transferase (GGT), Bile Acids (BA), TBIL (Total Bilirubin), Albumin (ALB), Blood Urea Nitrogen 371 (BUN), Total Cholesterol (CHOL) were quantified using a VetScan VS2 Chemistry Analyzer (Abaxis North America, USA).

### Blood Count Analysis

Mice were anesthetized and 20 μL of whole blood was collected via retro-orbital bleeding in lithium heparin blood collection tubes and 20 different hematologic parameters were measured using a Hemavet 950 FS blood count analyzer (Drew Scientific Group).

### Western Blotting

Mouse liver extracts were prepared by incubation in RIPA buffer (Invitrogen) containing protease inhibitors (Calbiochem, San Diego, CA). Protein was quantified by BCA assay (Thermo Scientific). Protein (40 mg or 80 mg for cytochrome C) was separated on 12% or 16% Tri-Glycine gels (Invitrogen) and transferred to Immobilon P membrane (0.2 μm pore size, Millipore). After 1 hour in phosphate-buffered saline-Tween (PBST) with 3% milk, membranes were incubated with antibodies to PPARGC1A(PGC1α) (Cat #NBP1-04676, Novus, 1:1000), SDHA XP (Cat #11998, Cell Signaling 1:1000), or Cytochrome C (Cat #4280, Cell Signaling 1:500), followed by secondary antibody conjugated to horseradish peroxidase (1:5000, Jackson Immune or Cell Signaling). Signal was revealed by ECL (Thermo) and imaged with a ChemiDoc MP imager (Bio-Rad). After detection, membrane was incubated with Ponceau S solution (Sigma) for 1 hr for normalization to loaded protein.

### Primary Human Hepatocytes

Experiments with primary human hepatocytes were performed by CN-Bio (Cambridge, UK). Briefly, primary human hepatocytes (PHHs), human Kupffer cells (HKs) and human stellate cells (HSCs) were seeded onto CN-Bio's PhysioMimix LC12 MPS culture plates at 6×10⁵ cells for PHHs and 6×10⁴ cells for HKs and HSCs in 1.6 ml of CN-Bio's HEP-lean media with 5% FCS. Throughout the experiment the cells were maintained at a flow rate of 1 μl/s. After 24 hours (Day 1) of seeding, the media was changed to HEP-lean media and the cells were incubated until day 4 to allow the cells to form microtissues. At day 4 post seeding, media was changed to HEP-fat media and treated with DMSO or NCT (5, 15, 40 μM). Media was replaced on days 6 and 8. Cells were harvested on day 10 for RNA extraction and culture media was collected for ELISA analysis.

### RT-PCR

Total RNA was isolated from liver tissues and primary human hepatocytes using Trizol (Invitrogen). cDNA was amplified using 3 μg of total RNA using qScript cDNA SuperMix (Quanta BioSciences, Beverly, MA, USA). Quantitative real time PCR (RT-PCR) analysis was performed using SYBR® Select Master Mix (Applied Biosystems) and an ABI 7900HT thermal cycler (Applied Biosystems, Thermo Fisher Scientific). Ct values were normalized to 18s rRNA and are expressed as fold change over samples from mice fed NC or cultured human hepatocytes without NCT.

### Fatty Acid Oxidation Assay

FAO in liver lysate was measured according to manufacturer's instructions using a calorimetric assay kit (Cat #E-141, Biomedical Research Service Centre, State University of New York, Buffalo, NY).

### Nicotinamide Adenine Dinucleotide Assay

Total NAD level in liver lysate was analyzed according to manufacturer's instructions using a calorimetric NAD+/NADH assay kit (Cat #MET-5014, Cell Biolabs, Inc. USA)

### Citrate Synthase Activity

Citrate synthase activity in liver homogenate was measured according to manufacturer's instructions using a calorimetry based MitoCheck Citrate Synthase Activity Assay Kit (Cat #701040, Cayman Chemicals, USA).

### BCA Assay

A bicinchoninic acid (BCA) protein assay was performed according to manufacturer's instructions using a kit from Thermo Scientific (Cat #23225). BCA assay was used for protein quantification for the FAO assay and Western blotting. Absorbance at 550 nm was determined using a plate reader.

### Nitric Oxide Assay

T6PNE cells were maintained in RPMI (5.5 mM glucose, Corning) supplemented with 10% fetal bovine serum (FBS, Sigma-Aldrich) and 1% penicillin-streptomycin (pen-strep, Gibco) in 5% CO₂ at 37° C. Cells were treated with 0.12 μM palmitate plus 0 or 15 μM NCT for 3 days in 10 cm plates and harvested with 500 μl PBS. For tissue specimens, snap frozen mouse liver was weighed and homogenized with PBS. NO was measured with the QuantiChrom Nitric Oxide Assay kit (D2NO-100, BioAssay Systems). Homogenized samples (150 μl) were processed for deproteination with 8 μl ZnSO₄ and 8 μl NaOH. For normalization, an aliquot of samples from T6PNE cells was taken for BCA assay before deproteination. Samples and standard from kit incubated with reagents for 20 min at 60° C. and measured OD 540 nM.

### Palmitate-BSA Complex

Palmitate (150 mM) (Sigma-Aldrich) was prepared in 50% ethanol and precomplexed with 15% fatty acid-free BSA (Research Organics, Cleveland, OH, USA) in a 37° C. water shaker. BSA447 precomplexed palmitate was used as a 12 mM stock solution for all assays with a final concentration of 0.12 mM palmitate in cell culture medium.

### Mitochondrial DNA Analysis

Quantification of mtDNA was performed as described (BCurr Protoc Mouse Biol 2017; 7:47-54). Snap frozen mouse liver was homogenized and total cellular DNA was extracted with a QIAamp DNA kit (Qiagen) followed b PCR with the primers below:

16S rRNA primers
FWD:
5′-CCGCAAGGGAAAGATGAAAGAC-3′
REV:
5′-TCGTTTGGTTTCGGGGTTTC-3′
ND1 primers
FWD:
5′-CTAGCAGAAACAAACCGGGC-3′
REV:
5′-CCGGCTGCGTATTCTACGTT-3′
HK2 primers
FWD:
5′-GCCAGCCTCTCCTGATTTTAGTGT-3′
REV:
5′-GGGAACACAAAAGACCTCTTCTGG-3

### Statistical Analysis

Data are presented as mean±SEM of three or more samples as indicated. Statistical significance was assessed using Student's f-test or ANOVA.

### Long Term Administration of NCT LED to Decreased Body Weight

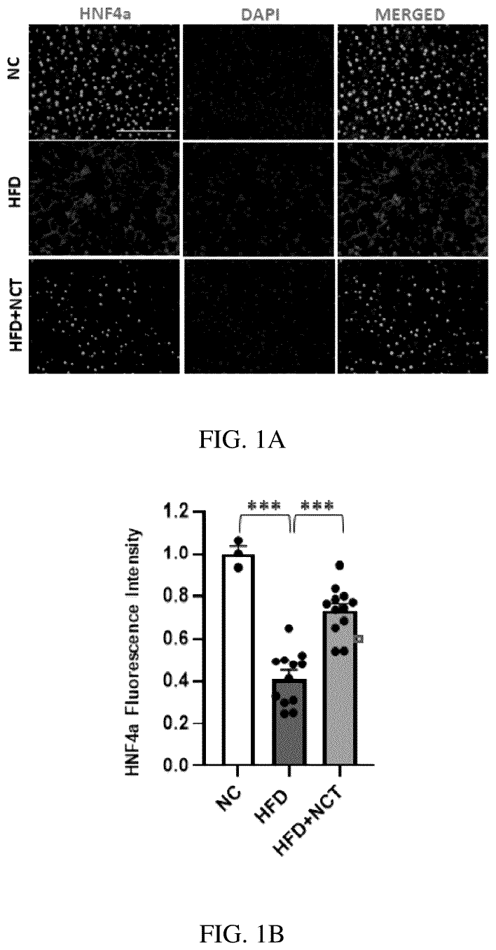

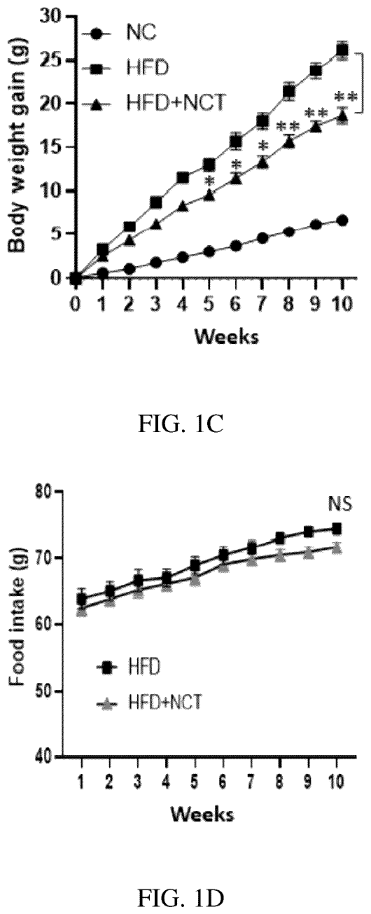

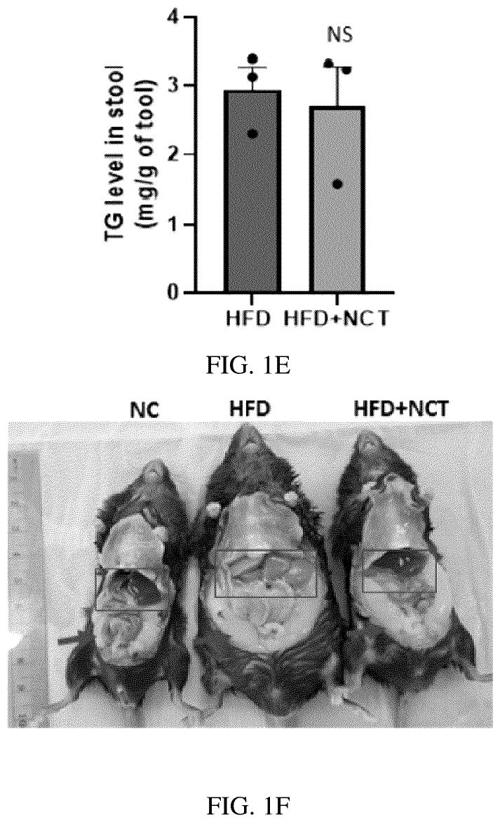

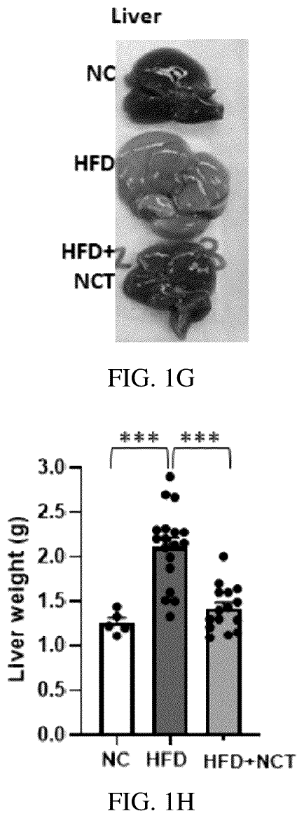

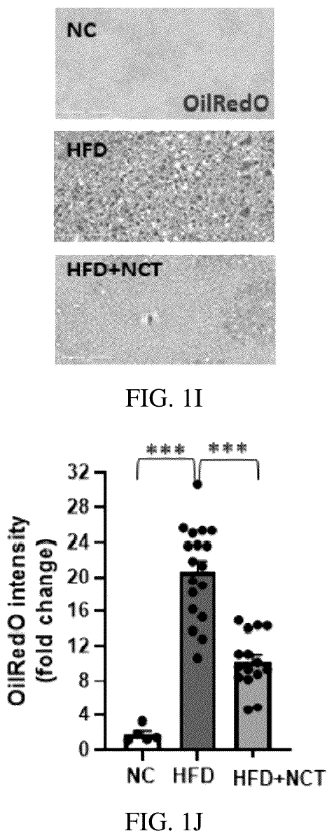

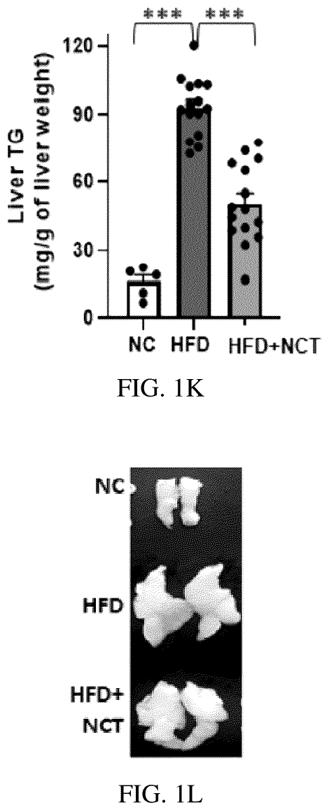

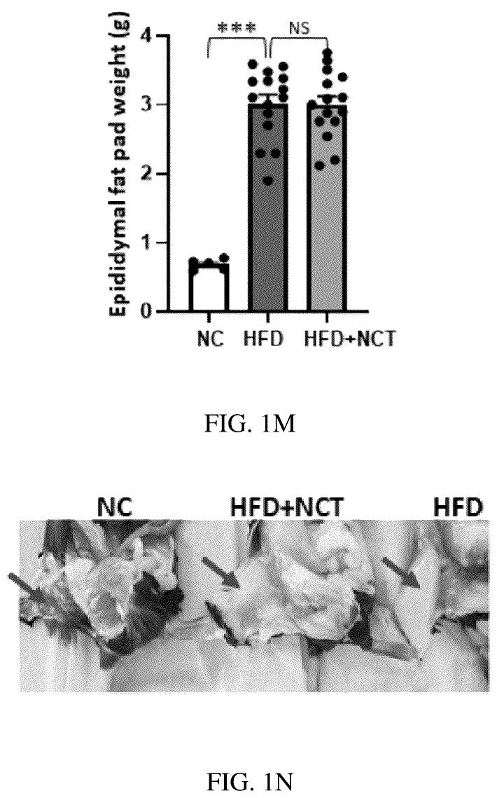

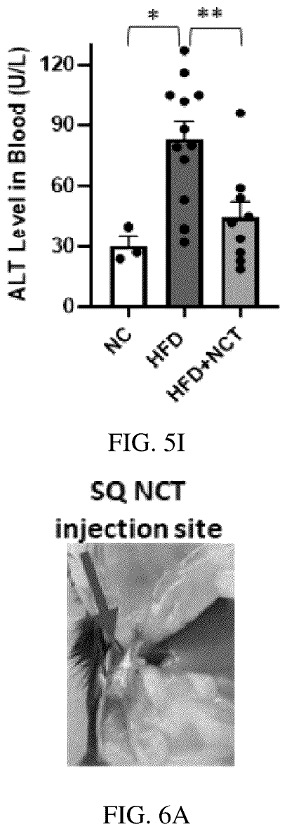

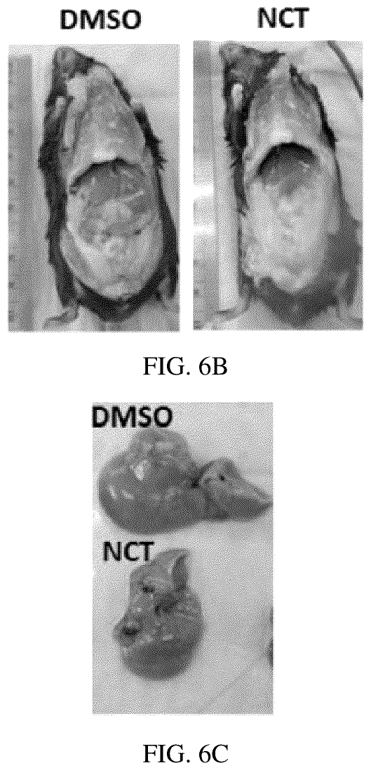

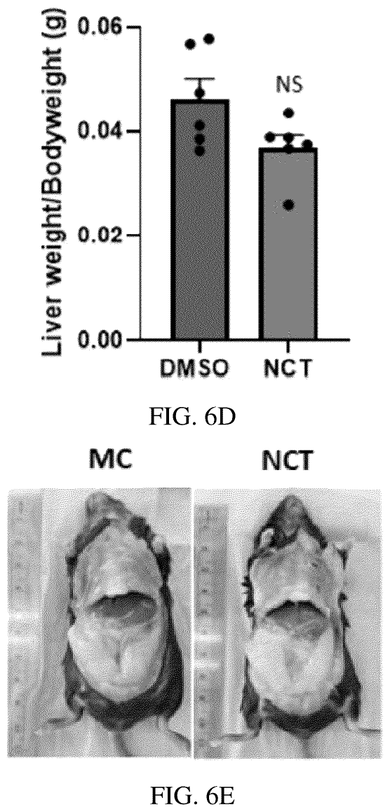

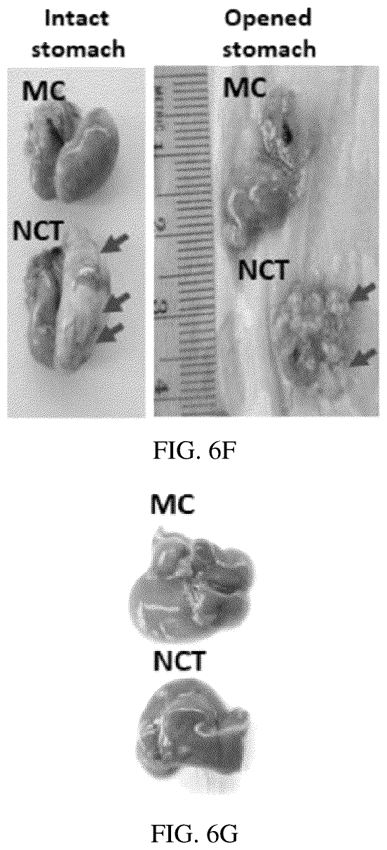

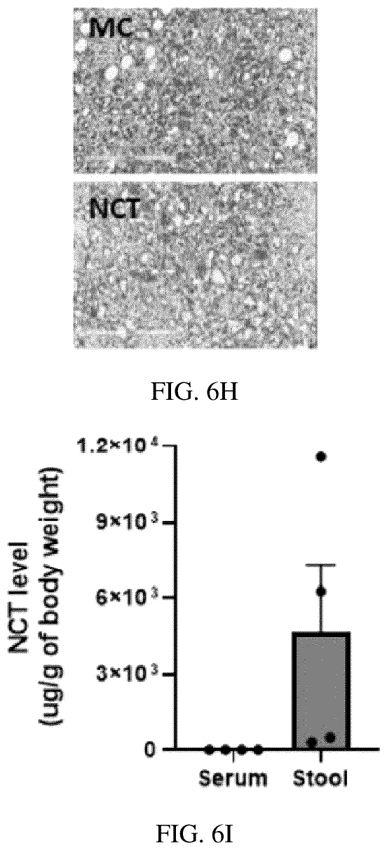

Previously, a short, two-week trial was conducted in which NCT, a potent HNF4α agonist, was administered to obese mice. NCT induced decreased hepatic steatosis due to stimulation of lipophagy, but there was no effect on body weight. In that study, NCT was administered intraperitoneally (IP) because HNF4α ligands, including the natural fatty acid ligands and the non-natural ligands that it has been studied are hydrophobic. That led to compound precipitation when it was attempted to deliver NCT subcutaneously (SQ) (FIGS. 6A-6D—NCT subcutaneous and oral gavage delivery led compound precipitation: 14 weeks old male C57BL/6 DIO mice were received 200 mg/kg of NCT 2 doses per day for 2 weeks via subcutaneous injection.) or by oral gavage (FIGS. 6E-I—14 weeks old male C57BL/6 DIO mice were received methyl cellulose (MC, vehicle control) or NCT (200 mg/kg) 2 doses per day via oral gavage.) for 2 weeks. Following oral gavage, NCT was present in stool but not serum (FIG. 6I) indicating malabsorption of compound that left the stomach (FIG. 6F). Given the desirability of oral administration, it was tested delivery of NCT mixed with high fat diet mouse chow (HFD) (Research Diets). HFD containing 4,000 ppm NCT (HFD+NCT) (Research Diets), was calculated to provide to a lean mouse weighing approximately 20 gm about the same dose of NCT (400 mg/kg/day) as was delivered using the IP administration protocol that it was used previously. HFD+NCT was administered to 6-week-old C57BL/6 male mice for ten weeks. FIGS. 1A-1N illustrates data from an experiment of long-term administration of N-trans-caffeoyltyramine led to reduced high fat diet induced weight gain and hepatic steatosis: C57BL/6 mice fed with normal chow (NC), HFD, or HFD+NCT during a 10 week study. To determine whether orally administered NCT was active, it was examined for effect on hepatic HNF4α expression, which it was found previously to be increased by NCT. HFD led to decreased expression of HNF4α and, consistent with the previous results in which NCT was administered IP, this was reversed by NCT (FIGS. 1A and 1i). Thus, oral administration of NCT at approximately the same dose as IP administration was effective at stimulating HNF4α activity.

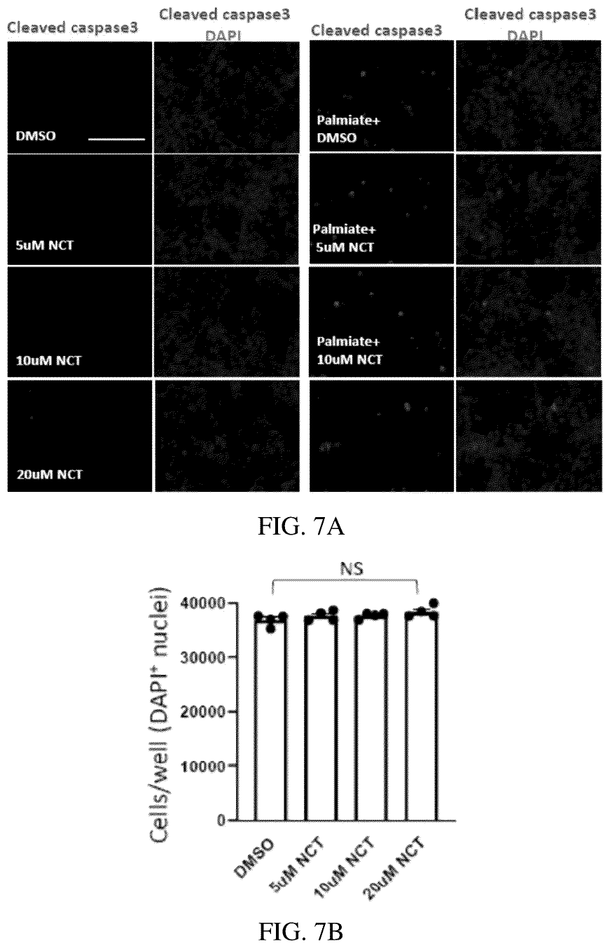

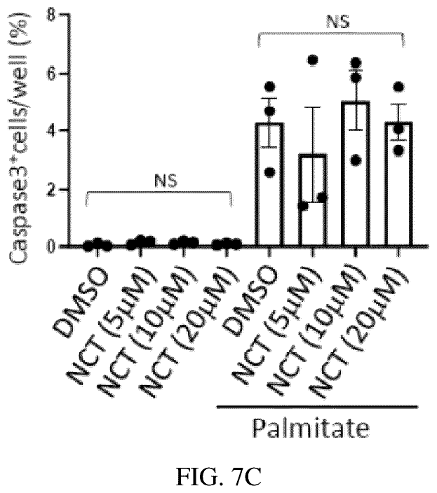

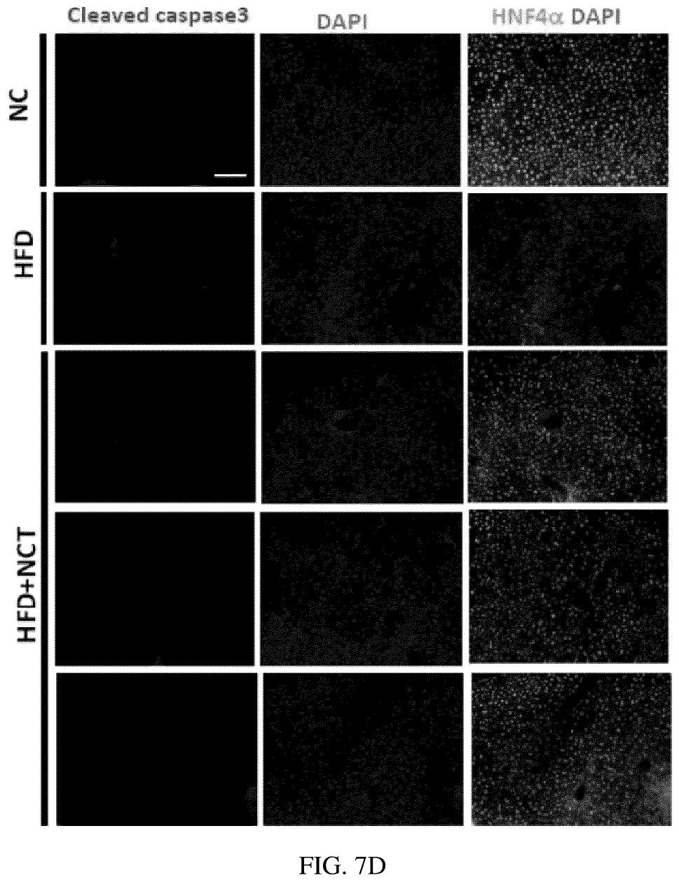

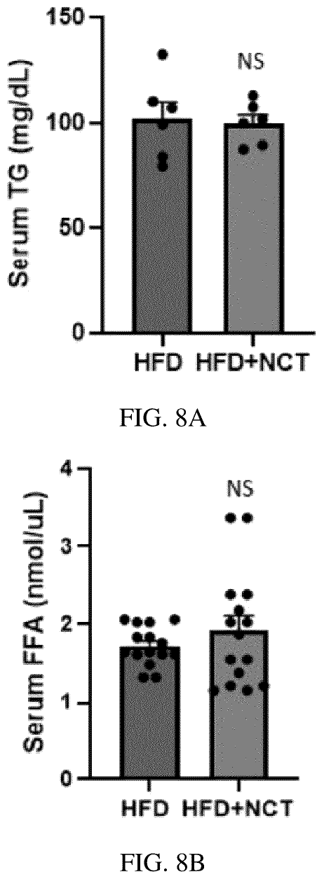

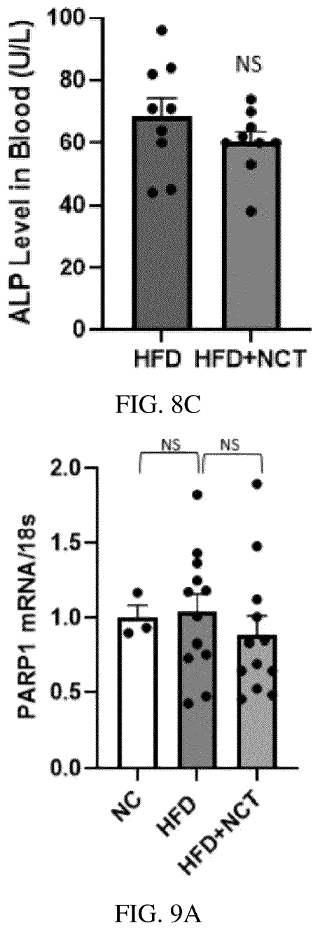

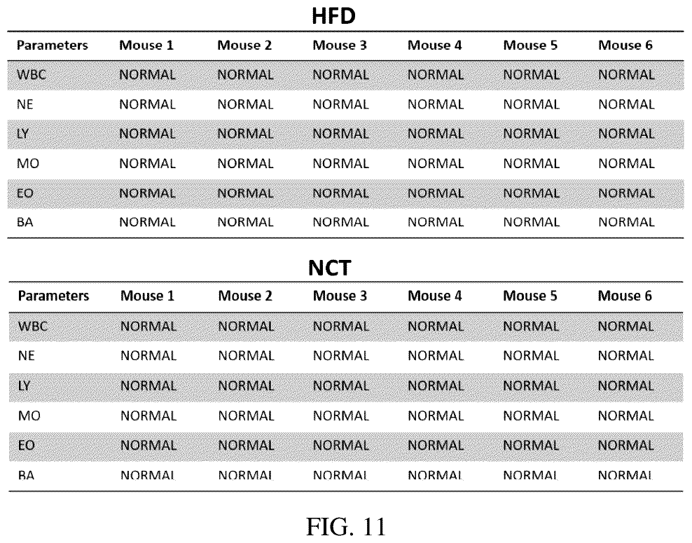

In striking contrast to the previous experiments with 2 weeks of IP NCT administration where any effect of NCT on body weight was not observed, it became evident at week 5 that mice fed HFD+NCT weighed less than mice fed HFD alone (FIG. 1C). By the end of the experiment at 10 weeks, the mice fed HFD+NCT weighed ˜10 gm less than the mice fed HFD, a 35-40% difference in body weight. One possible explanation for lower weight was that the mice consumed less chow, possibly due to long term toxicity of NCT. There was no discernible difference in physical activity or other behaviors between mice fed HFD and mice fed HFD+NCT (FIG. 10). Caspase 3, a marker of apoptosis, was low in mice fed HFD, and there was no change with NCT (FIGS. 7A-7D—T6PNE cells were treated for 3 days with or without palmitate (0.1 mM) and 0, 5, 10, 20 μM NCT, followed by fixing with 4% PFA and immunostaining for cleaved caspase3 and DAPI. Quantification was with a Celigo imaging cytometer (Nexcelom Bioscience)). There was no difference in the amount of HFD chow and HFD+NCT chow that was consumed, indicating that the NCT was not aversive and did not cause the mice to become ill and consume less chow (FIG. 1D). Stool triglyceride (TG) was equal in the HFD and HFD+NCT groups, ruling out the possibility of fat malabsorption as being responsible for the difference in weight (FIG. 1E). The HFD+NCT mice were markedly less obese (FIG. 1F), with substantially less subcutaneous fat (FIG. 1N) but no change in epididymal fat pad weight at the end of the study (FIG. 1L and FIG. 1M). HFD+NCT mice had redder livers (FIG. 1G), and lower liver weight (FIG. 1H). Consistent with the increased redness and decreased weight, the HFD+NCT livers exhibited decreased Oil Red O staining (FIG. 11, quantified in FIG. 1J), and lower TG (FIG. 1K). There was no difference in circulating TG or free fatty acid (FFA) (FIGS. 8A-8C—NCT did not modulate the TG, FFA and ALP level in the circulation).

NCT LED to Increased Fatty Acid Oxidation (FAO) in the Livers of Obese Mice with Hepatic Steatosis but this was a Secondary Rather than Primary Effect

The large weight difference between the HFD and HFD+NCT groups in conjunction with decreased subcutaneous and hepatic adiposity was striking. There are only two routes of fat elimination; in the stool by malabsorption, which it was ruled out (FIG. 1E) and by oxidation. Thus, it was hypothesized that NCT was inducing an increase in FAO. Quantification of FAO was done using an assay that measures NADH by the conversion of iodonitrotetrazolium (INT) to INT-Formazan mediated by the NADH-requiring enzyme diaphorase. Performing the assay in the presence and absence of added octanoyl-CoA, which is converted through fatty acid β-oxidation to acetyl-CoA with conversion of NAD+ to NADH, provides specificity for FAO. The level of fatty acid oxidation is determined as the INT-formazan level in the presence of octanoyl CoA minus the level in the absence of octanoyl CoA.

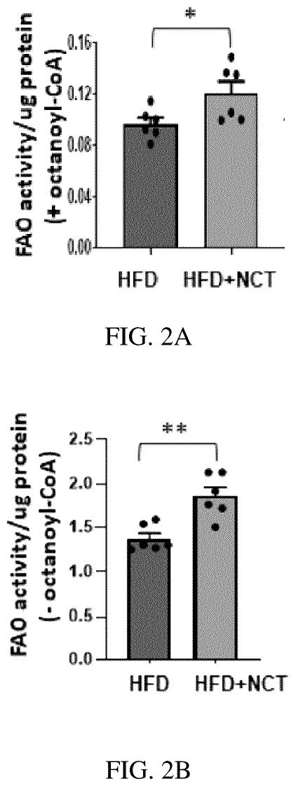

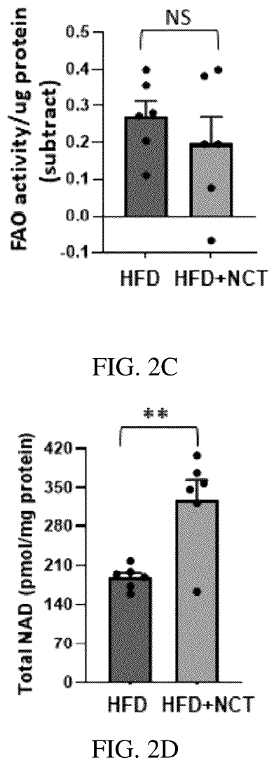

NCT induced an increase in FAO activity in the presence of octanoyl CoA (FIG. 2A). However, there was also an increase in the baseline activity in the absence of octanoyl CoA (FIG. 2B) so that the overall activity in the assay was unchanged (FIG. 2C). Because this assay is sensitive to the cellular mitochondrial mass, this suggested that the effect of NCT might be on mitochondrial mass rather than specifically on fatty acid oxidation, i.e., NCT might be acting to stimulate increased mitochondrial mass with a consequent increase in total fatty acid oxidation without stimulating an increase in the level of fatty acid oxidation per unit of mitochondrial mass. Independent measurement of total cellular NAD, the majority of which is in the mitochondria, revealed a substantial increase in the livers of mice treated with NCT (FIG. 2D). Thus, the effect of NCT on FAO appeared to be a secondary rather than primary effect.

### NCT Induced an Increase in Mitochondrial Mass and Reduction in Mitochondrial Stress

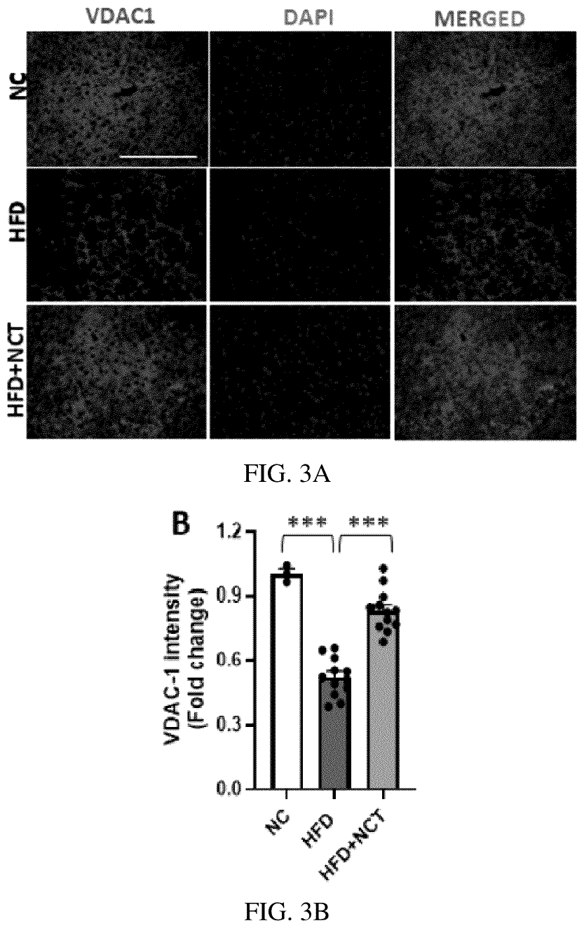

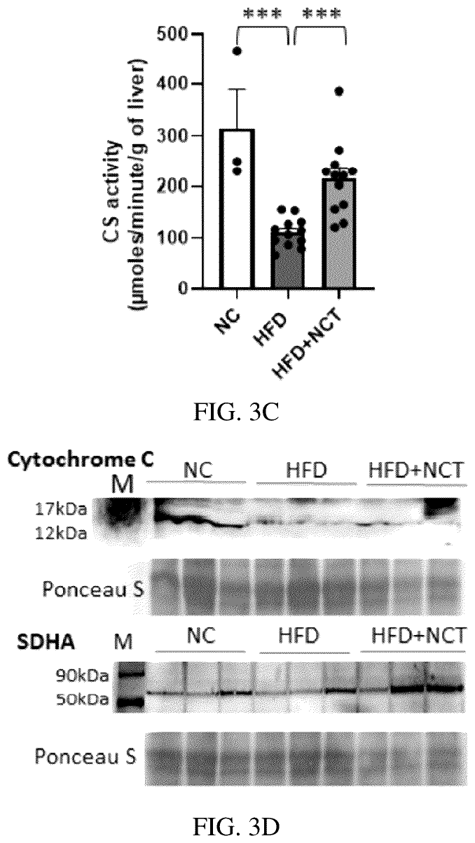

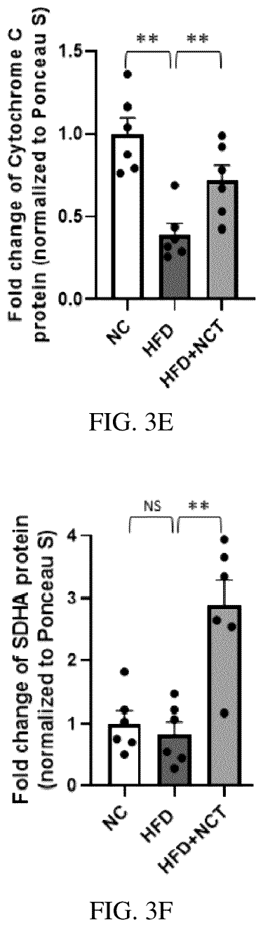

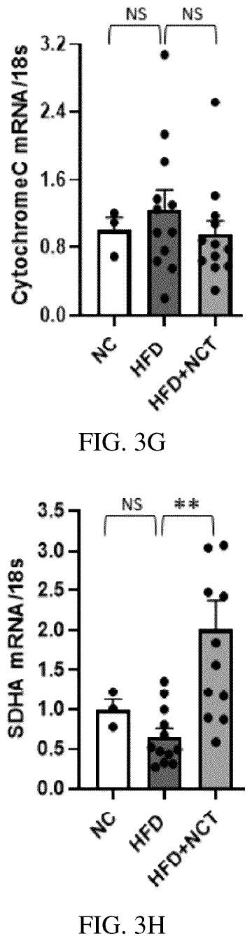

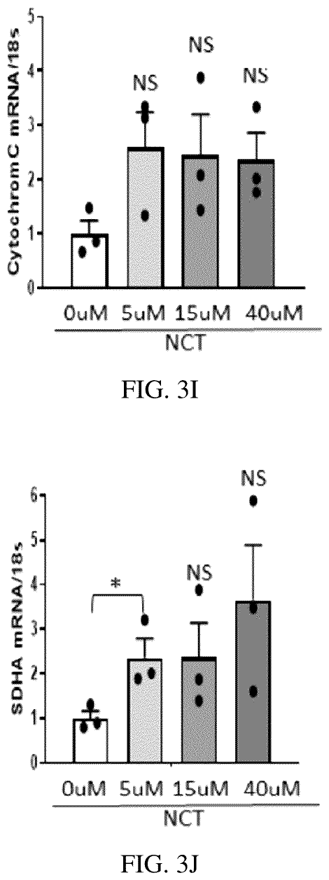

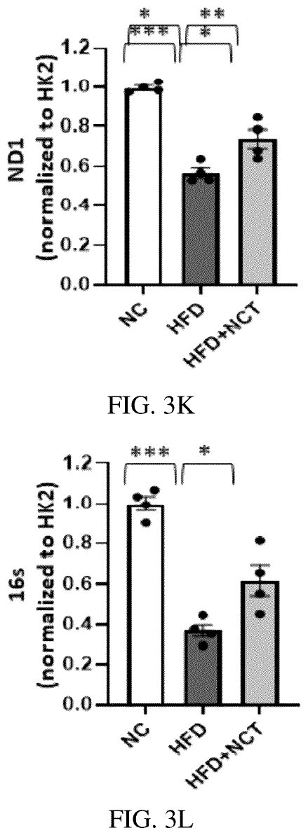

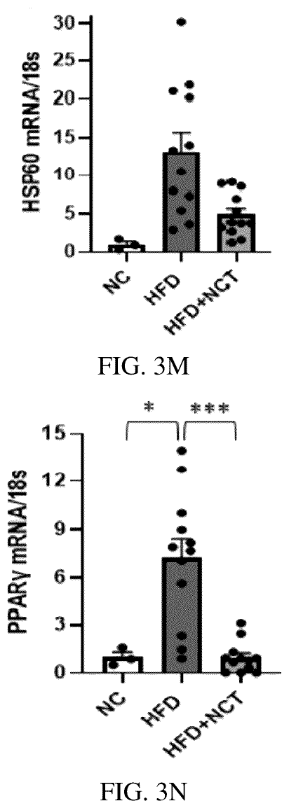

Based on the FAO oxidation result and increased total cellular NAD, study markers of mitochondrial mass were determined directly whether NCT effected an increase. The two most commonly used protein markers of mitochondrial mass are VDAC1, which is located on the outer mitochondrial membrane (11), and citrate synthase, which is located in the mitochondrial matrix (12). HFD led to a large decrease in VDAC1 expression and citrate synthase activity, with substantial reversal of those decreases by NCT (FIGS. 3A-C).

The major function of mitochondria is oxidative phosphorylation. To determine whether the increased mitochondrial mass was reflected in increased expression of proteins involved in oxidative phosphorylation, was measured cytochrome C and succinate dehydrogenase expression, both of which are important components of the respiratory. Cytochrome C plays a dual role: in mitochondria, being critical for mitochondrial respiration but also playing a role in cell survival. Succinate dehydrogenase, encoded by the SDHA gene, is the catalytic subunit of succinate-ubiquinone oxidoreductase, a complex of the mitochondrial respiratory chain. HFD decreased hepatic cytochrome C and succinate dehydrogenase protein levels and this was reversed by NCT (FIGS. 3D-F). SDHA but not cytochrome C mRNA was increased by NCT in mouse liver (FIG. 3G and FIG. 3H). This was reflected in studies with primary human hepatocytes, where NCT increased the level of SDHA but not cytochrome C mRNA (FIG. 3I and FIG. 3J).

An increase in mitochondrial proteins could be due to an increase in the number of mitochondria or simply to an increase in mitochondrial size. Thus, the mitochondrial DNA content was measured, finding that NCT induced a significant increase in mitochondrial DNA, as measured by the level of DNA encoding the mitochondrial genes ND1 and 16S rRNA (FIG. 3K and FIG. 3L).

An important feature of NAFLD and its progression to NASH is mitochondrial stress. To determine whether the reduction in hepatic steatosis and increase in mitochondrial mass induced by NCT translated to 167 reduced mitochondrial stress, the expression of HSP60 was examined, a mitochondrial chaperone that is induced by mitochondrial stress, including HFD (FIG. 3M). The level of HSP60 mRNA was greatly decreased by NCT, demonstrating alleviation of mitochondrial stress (FIG. 3M). Similarly, PPARγ plays an important role in the mitochondrial stress response, being activated by fatty acids and exhibiting high expression in fatty liver disease. NCT administration dramatically reduced PPARγ expression to the level in the livers of mice fed normal chow (FIG. 3N).

### NCT Increased the Activity of the PPARGC1A Pathway

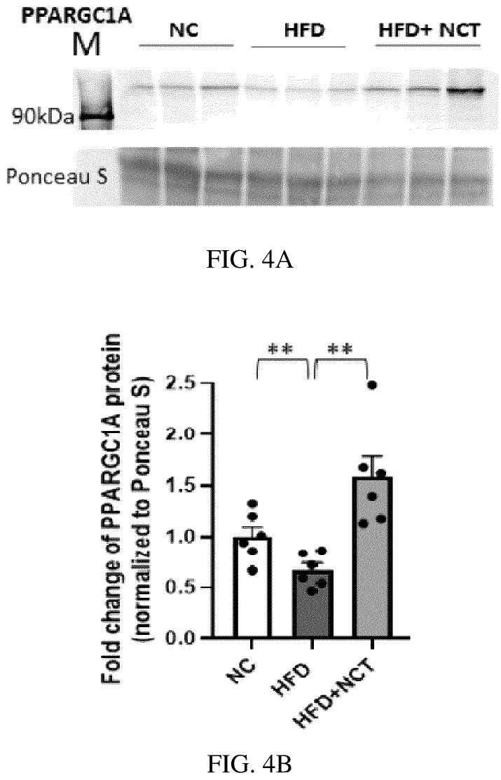

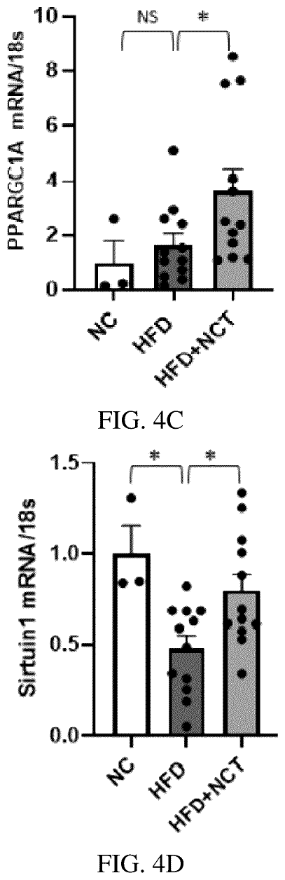

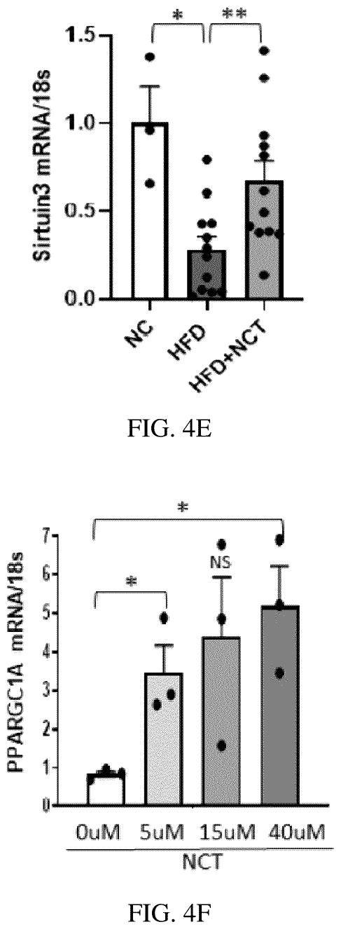

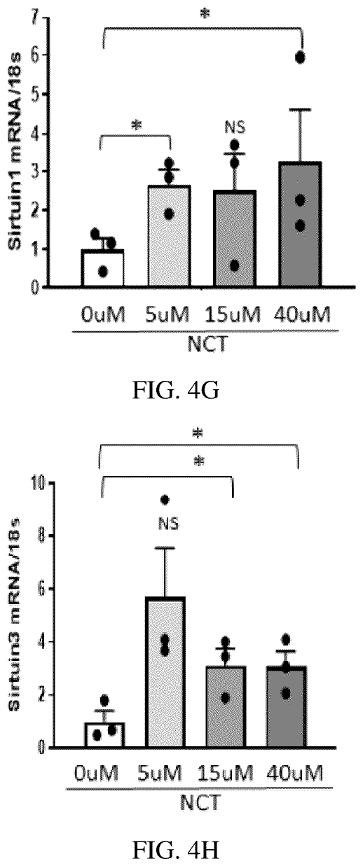

The ability of NCT to increase mitochondrial mass raised the question of the mechanism by which that occurred. Examination of RNA-seq data from the livers of mice treated for 2 weeks with NCT revealed an approximately 6-fold increase in PPARGC1A (PGC1α) mRNA (GEO 174848). PPARGC1A plays an important role in mitochondrial biogenesis but is not known to be regulated by HNF4α. After ten weeks of oral NCT administration, PPARGC1A protein and mRNA were increased (FIGS. 4A-C) in mouse liver. The PPARGC1A mRNA level in primary human hepatocytes cultured with NCT was also increased (FIG. 4F).

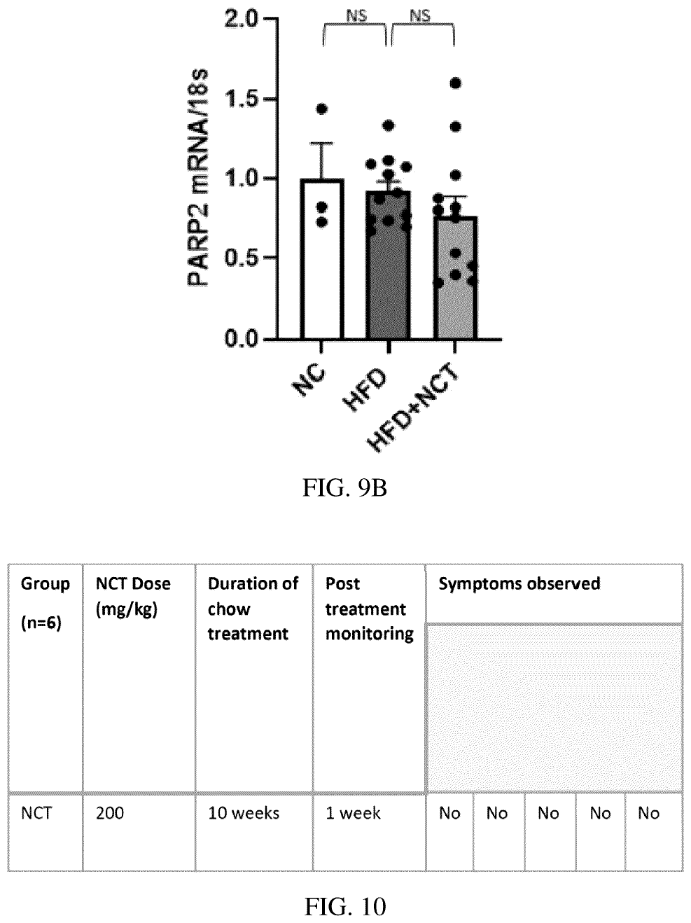

PPARGC1A activity is controlled by sirtuins, which are NAD-dependent deacetylases. They are both downstream targets of transcriptional activation by PPARGC1A and activators of PPARGC1A activity through deacetylation. HFD reduced Sirt1 and Sirt3 mRNA levels, consistent with previous studies. NCT almost completely reversed the effect of HFD on sirtuin gene expression in mouse liver (FIG. 4D and FIG. 4E) and had a significant effect in primary human hepatocytes (FIG. 4G and FIG. 4H). Poly(ADP-ribose) polymerases (PARPs) inhibit mitochondrial function and PPARGC1A activity. In contrast to sirtuin expression, Parp1 and Parp2 mRNA levels were not modulated by NCT (FIG. 9A-9B—No difference in Parp1 and Parp2 mRNA expression).

### NCT Inhibited Inflammation

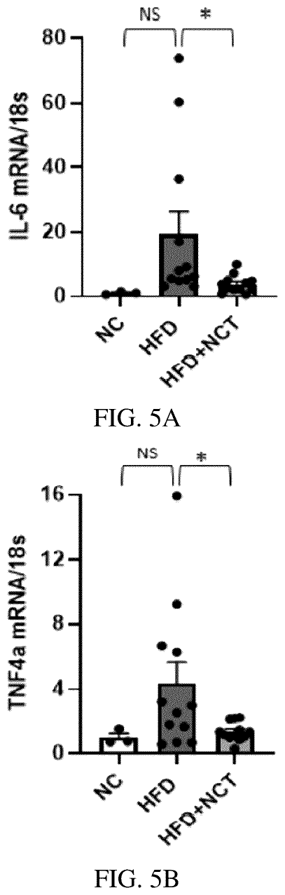

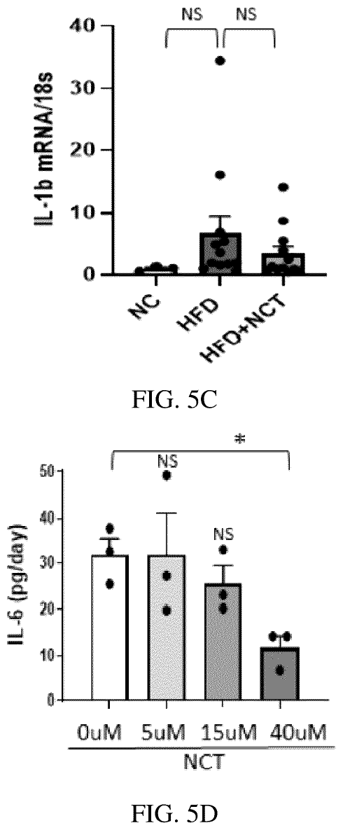

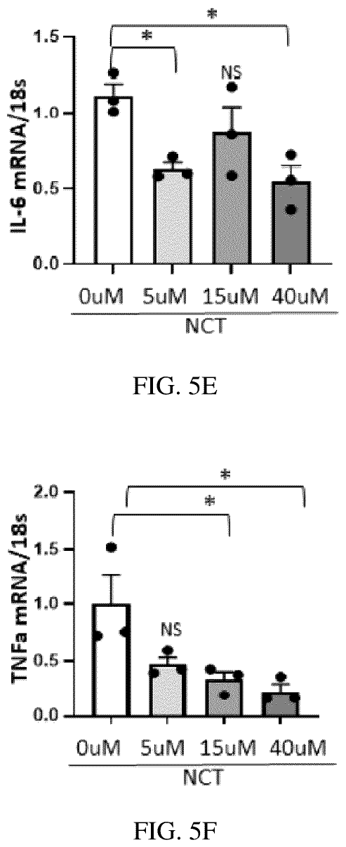

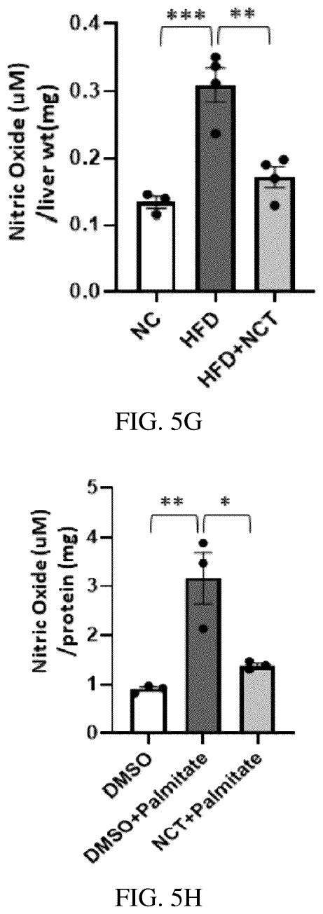

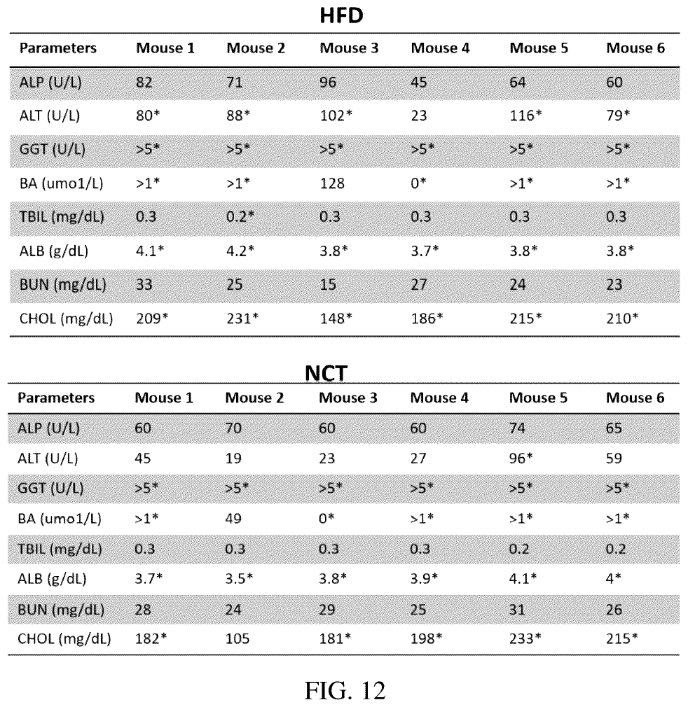

Inflammation is a major factor in the pathophysiology of NAFLD and its progression to NASH. Inflammation contributes to essential features of NASH pathology, including fibrosis and hepatocyte death, ultimately leading to cirrhosis. IL-6 and TNFα are inflammatory mediators that are important in NASH. Both were significantly reduced by NCT in the livers of HFD+NCT mice compared to HFD mouse livers (FIG. 5A and FIG. 5B). Similarly, IL-6 mRNA and protein were reduced by NCT in cultured human hepatocytes (FIG. 5D and FIG. 5E). TNFα mRNA was also decreased in a dose-responsive manner by NCT in cultured human hepatocytes (FIG. 5F) but ILI p expression was not significantly changed (FIG. 5C). Nitric oxide (NO) plays an important role in inflammatory responses, including NAFLD. NO was increased in the livers of mice fed HFD (FIG. 5G) as well as in cultured cells treated with palmitate (FIG. 5H) and was decreased by NCT in both settings (FIG. 5G and FIG. 5H). The blood ALT level was elevated in mice fed HFD and was significantly decreased by NCT (FIG. 5I). The blood ALP was unchanged (FIG. 8C). Other components of the liver profile panel and hematological analysis showed no difference in HFD versus HFD+NCT mice (FIG. 11 and FIG. 12).

The principal finding of the studies presented here is that activating HNF4α with the potent HNF4α agonist NCT led to decreased weight in mice fed a high fat diet. This was due to a substantial increase in mitochondrial mass and consequent increase in fatty acid oxidation. The earlier studies where NCT was administered to mice for a shorter time found a different effect, i.e., stimulation of lipophagy. That led to decreased hepatic steatosis but no change in weight, as the fatty acids released from lipid vesicle by lipophagy appeared to redistribute to adipose tissue. Thus, there appear to be at least two independent effects of HNF4α agonism on fat storage: an immediate effect on hepatic steatosis through lipophagy and a longer-term effect on mitochondrial mass leading to increased mitochondrial function, including fatty acid oxidation.

A key to uncovering the effect of pharmacological activation of HNF4α on mitochondrial mass was the finding that oral delivery of NCT in high fat chow was effective, permitting long term delivery of the compound. Being able to deliver drugs orally is often critical to achieving effective clinical translation, so this was an important goal. Oral delivery is particularly important for highly prevalent and chronic diseases like NAFLD/NASH. The previous study of NCT used IP delivery because the compound is hydrophobic, limiting some routes of delivery. However, by combining NCT with high fat chow, it was demonstrated that efficacy in a HFD diet mouse model of NAFLD. Of note, there is a large difference between rodent and human microsomal stability of NCT, with NCT being much more stable in the presence of human microsomes than with rodent. While this bodes well for eventual use in humans, PK studies in the context of human clinical trials will be required. A potentially important consideration is that it is likely that NCT and fatty acids are in competition for the HNF4α ligand binding pocket. The concentration of fatty acids, which act as HNF4α antagonists, in the nucleus, is unknown, but if it is related to the overall level of steatosis in a particular organ, that could affect the dose required for efficacy.

The results led to 234 the increase in mitochondrial mass by the decreased weight in the mice fed HFD+NCT and those fed HFD alone. Since food consumption was the same and there was no fat malabsorption, only an increase in fatty acid oxidation could reasonably account for the lower weight of the HFD+NCT mice. This was borne out by increased oxidation of octanoyl CoA in mice fed HFD+NCT. However, the increase in fatty acid oxidation was not selective, as there was increased NADH production even in the absence of octanoyl CoA. Consistent with a general increase in mitochondrial mass and function, VDAC1 and citrate synthase were increased, as were the oxidative phosphorylation components cytochrome C and SDHA. Consistent with a general increase in mitochondrial function, total cellular NAD was substantially increased by NCT.

The pathways controlling mitochondrial mass are complex and interlocking and a protein at the center of those pathways is the transcriptional coactivator PPARGC1A (PGC1α). PPARGC1A interacts with multiple factors involved in mitochondrial biogenesis and function. Notably, it interacts directly with HNF4α in regulating gene expression, particularly gluconeogenesis (26), but HNF4α has not heretofore been recognized as having an effect on mitochondrial biogenesis. PPARGC1A expression was increased by NCT but is not thought to be a direct HNF4α target based on ChIP-seq data. Moreover, NCT increased the expression of sirtuins, which control the level of PPARGC1A activity through deacetylation. Pharmacological enhancement of mitochondrial mass and function has been a long-sought goal, but there have been no robustly active compounds with the desired activity. A major motivation for the discovery of approaches to increase mitochondrial mass and function is that mitochondrial function is declines with aging. Given the finding of increased mitochondrial mass and function by HNFα activation NCT has the potential to be of benefit in that setting. However, a consideration with stimulating HNF4α activity is that it is expressed at a high level only in a subset of tissues, including the liver, intestine, pancreas, and kidney. Thus, effects of HNF4α agonists may be limited to those organs, although there is evidence for low level HNF4 a expression elsewhere. Furthermore, despite the fact that HNF4α is not expressed in adipocytes, the effect of NCT extended to decreased adiposity. Furthermore, there are important age-related diseases that affect organs expressing HNF4α. Most prominent among those is type 2 diabetes, in which age and lipotoxic effects on pancreatic p-cells over time are critical. Thus, NCT has potential as a type 2 diabetes therapeutic. The liver plays a key role in type 2 diabetes, where the predominant paradigm is that cellular stress and inflammation contribute to insulin resistance and dysregulated hepatic gluconeogenesis, as well as being important in the progression from NAFLD to NASH. NCT was effective at reducing cellular stress and inflammation, supporting a role in multiple disease in which those factors are central.

Of significance for eventual clinical translation of NCT, has not seen any deleterious effects of this compound in any of the studies, including a short-term maximum tolerated dose study, two-week IP administration at a fairly high dose of 200 mg/kg bid, and long term oral administration. Given the chronic nature of disorders of lipotoxicity, the lack of discernable toxicity of NCT is critical, but this will obviously require additional investigation in human clinical trials.

What is claimed is:

1. A method for enhancing mitochondrial mass and function in a subject in need, the method comprising:
administering to the subject in need thereof an oral composition comprising at least one carrier and an effective amount of a compound of Formula (I), 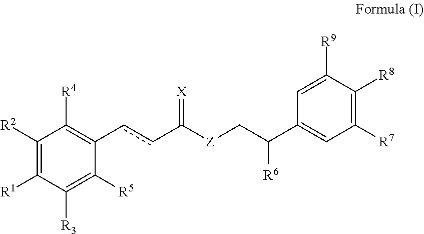
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl
wherein the subject is on a high fat diet or high calorie diet.

2. The method of claim 1, wherein the subject is administered the oral composition for at least 6 weeks.

3. The method of claim 1 or 2, wherein the subject's body weight is reduced by from about 2% to about 20%.

4. The method of claim 1 or 2, wherein the oral composition is formulated as a dietary supplement, food ingredient or additive, a medical food, nutraceutical or pharmaceutical composition.

5. The method of claim 1 or 2, wherein the compound of Formula (I) is selected from the group consisting of N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine.

6. The method of claim 1 or 2, wherein the subject is administered from about 10 mg to about 120 mg of a compound of the oral composition.

7. The method of claim 1 or 2, wherein the oral composition is formulated as a tablet or capsule.

8. The method of claim 1 or 2, wherein the carrier is selected from at least one of a sugar, a starch, cellulose, powdered tragacanth, malt, gelatin, talc, excipient, oil, glycol, polyol, ester, agar, buffering agent, alginic acid, isotonic saline, ethyl alcohol, pH buffered solutions, or polyesters.

9. The method of claim 1 or 2, wherein the compound of Formula (I) comprises about 0.1% to about 99% of the composition.

10. The method of claim 1 or 2, wherein the oral composition is formulated as a dietary supplement, food ingredient or additive, a medical food, nutraceutical, or pharmaceutical.

11. The method of claim 1 or 2, wherein the oral composition further comprises a preservative.

12. The method of claim 11, wherein the preservative is from about 0.01% to about 5% by weight of the composition.

13. The method of claim 1 or 2, wherein the oral composition has a pH between 2 and 7.4.

14. The method of claim 1 or 2, wherein the oral composition is formulated as a liquid.

15. A method for increasing fatty acid oxidation in a subject in need, the method comprising:
administering to the subject in need thereof an oral composition comprising at least one carrier and an effective amount of a compound of Formula (I) 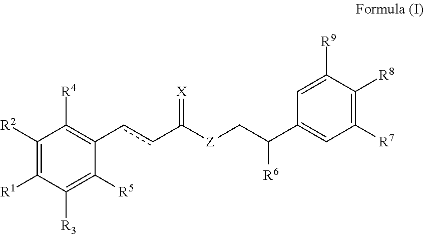
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆ alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl
wherein the subject is on a high fat diet or high calorie diet.

16. The method of claim 15, wherein the subject is administered the oral composition for at least 6 weeks.

17. The method of claim 15 or 16, wherein the subject's body weight is reduced by from about 2% to about 20%.

18. The method of claim 15 or 16, wherein the oral composition is formulated as a dietary supplement, food ingredient or additive, a medical food, nutraceutical or pharmaceutical composition.

19. The method of claim 15 or 16, wherein the compound of Formula (I) is selected from the group consisting of N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine.

20. The method of claim 15 or 16, wherein the subject is administered from about 10 mg to about 120 mg of a compound of the oral composition.

21. The method of claim 15 or 16, wherein the oral composition is formulated as a tablet or capsule.

22. The method of claim 15 or 16, wherein the carrier is selected from at least one of a sugar, a starch, cellulose, powdered tragacanth, malt, gelatin, talc, excipient, oil, glycol, polyol, ester, agar, buffering agent, alginic acid, isotonic saline, ethyl alcohol, pH buffered solutions, or polyesters.

23. The method of claim 15 or 16, wherein the compound of Formula (I) comprises about 0.1% to about 99% of the composition.

24. The method of claim 15 or 16, wherein the oral composition is formulated as a dietary supplement, food ingredient or additive, a medical food, nutraceutical, or pharmaceutical.

25. The method of claim 15 or 16, wherein the oral composition further comprises a preservative.

26. The method of claim 25, wherein the preservative is from about 0.01% to about 5% by weight of the composition.

27. The method of claim 15 or 16, wherein the oral composition has a pH between 2 and 7.4.

28. The method of claim 15 or 16, wherein the oral composition is formulated as a liquid.

29. A method for treating fatty acid excess in a subject in need, the method comprising:
administering to the subject in need thereof an oral composition comprising at least one carrier and an effective amount of N-trans-caffeoyltyramine,
wherein the subject is on a high fat diet or high calorie diet.

30. The method of claim 29, wherein the subject is administered the oral composition for at least 6 weeks.

31. The method of claim 29 or 30, wherein the subject's body weight is reduced by from about 2 to about 20%.

32. The method of claim 29 or 30, wherein the oral composition is formulated as a dietary supplement, food ingredient or additive, a medical food, nutraceutical or pharmaceutical composition.

33. A method for reducing one or more signs of aging in a subject in need, the method comprising:
administering to the subject in need thereof an oral composition comprising at least one carrier and an effective amount of N-trans-caffeoyltyramine,
wherein the subject is on a high fat diet or high calorie diet.

34. The method of claim 33, wherein the subject is administered the oral composition for at least 6 weeks.

35. The method of claim 33 or 34, wherein the subject's body weight is reduced by from about 2 to about 20%.

36. The method of any one of claims 33 to 35, wherein the oral composition is formulated as a dietary supplement, food ingredient or additive, a medical food, nutraceutical or pharmaceutical composition.

37. A method for increasing cellular energy in a subject in need, the method comprising:
administering to the subject in need thereof an oral composition comprising at least one carrier and an effective amount of N-trans-caffeoyltyramine,
wherein the subject is on a high fat diet or high calorie diet.

38. The method of claim 37, wherein the subject is administered the oral composition for at least 6 weeks.

39. The method of claim 37 or 38, wherein the subject's body weight is reduced by from about 2 to about 20%.

40. The method of any one of claims 37 to 39, wherein the oral composition is formulated as a dietary supplement, food ingredient or additive, a medical food, nutraceutical or pharmaceutical composition.

41. A method for enhancing energy in a subject in need, the method comprising:
administering to the subject in need thereof an oral composition comprising at least one carrier and an effective amount of N-trans-caffeoyltyramine,
wherein the subject is on a high fat diet or high calorie diet.

42. The method of claim 41, wherein the subject is administered the oral composition for at least 6 weeks.

43. The method of claim 41 or 42, wherein the subject's body weight is reduced by from about 2 to about 20%.

44. The method of any one of claims 41 to 43, wherein the oral composition is formulated as a dietary supplement, food ingredient or additive, a medical food, nutraceutical or pharmaceutical composition.

45. A method for increasing NAD in a subject in need thereof, the method comprising:
administering to the subject in need thereof an oral composition comprising at least one carrier and an effective amount of N-trans-caffeoyltyramine,
wherein the subject is on a high fat diet or high calorie diet.

46. The method of claim 45, wherein the subject is administered the oral composition for at least 6 weeks.

47. The method of claim 45 or 46, wherein the subject's body weight is reduced by from about 2 to about 20%.

48. The method of any one of claims 45 to 46, wherein the oral composition is formulated as a dietary supplement, food ingredient or additive, a medical food, nutraceutical or pharmaceutical composition.

49. A method for decreasing muscle atrophy in a subject in need thereof, the method comprising:
administering to the subject in need thereof an oral composition comprising at least one carrier and an effective amount of a compound of Formula (I), 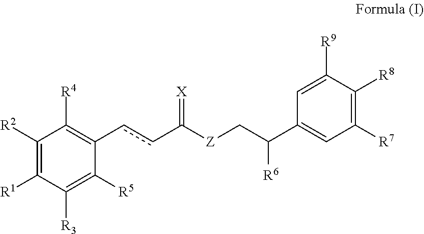
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

50. A method for increasing performance recovery in a subject in need thereof, the method comprising:
administering to the subject in need thereof an oral composition comprising at least one carrier and an effective amount of a compound of Formula (I), 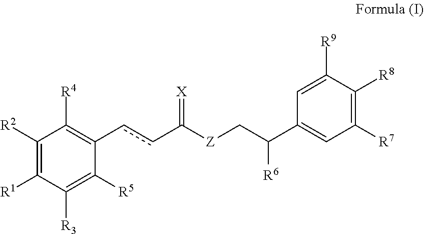
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.
